(19) United States
(12) Patent Application Publication
(10) Pub. No.: US 2022/0139535 A1
(43) Pub. Date: May 5, 2022

(54) EFFICIENT DETERMINATION OF A DATA ENTITY STORING HEALTHCARE DATA THROUGH MAPPED ENTRY AND/OR TRAVERSAL OF A SEMANTIC DATA STRUCTURE

(71) Applicant: MapViser Medical LLC, Moraga, CA (US)

(72) Inventors: Nataliya M. KUSHNIR, Moraga, CA (US); Dmitriy A. SUKHOV, Moraga, CA (US); Ivan SUKHORYABA, Kherson (UA); Dmytro Ukhan, Kherson (UA); Dmytro CHORNOMORCHENKO, Kherson (UA)

(73) Assignee: MapViser Medical LLC, Moraga, CA (US)

(21) Appl. No.: 17/089,702

(22) Filed: Nov. 4, 2020

(51) Int. Cl. G16H 40/20; G16H 10/60; G16H 50/30; G16H 50/70; G16H 70/60; G16H 70/20; G06F 16/22; G06F 16/28; G06F 16/245; G06F 21/62; G06N 3/04; G06N 3/08

(52) U.S. Cl. CPC G16H 40/20; G16H 10/60; G16H 50/30; G16H 50/70; G16H 70/60; G16H 70/20; A61B 5/1112; G06F 16/285; G06F 16/245; G06F 21/6245; G06N 3/04; G06N 3/08; G06F 16/2228

(57) ABSTRACT

Disclosed are a method, a device, and/or a system of efficient determination of a data entity storing healthcare data through mapped entry and/or traversal of a semantic data structure. In one embodiment, a method includes generating an entry criteria data for calculation of an entry point into one or more semantic data structure nodes. The semantic data structure is queried to receive unique identifiers of a plurality of data entities. A tag data matching the entry criteria is mapped from a term value of a medical taxonomy. The plurality of data entities includes a service profile, a professional profile, and/or a technology profile. A card data for each data entity associated with the unique identifiers is transmitted to a client device. A profile request may be generated and the semantic data structure traversed for related data entities to increase probability of connection to a solution profile applicable to the user.

### FIELD OF TECHNOLOGY

This disclosure relates generally to data processing devices and, more particularly, to a method, a device, and/or a system of efficient determination of a data entity storing healthcare data through mapped entry and/or traversal of a semantic data structure.

### BACKGROUND

Data structures stored as electronic information on computer readable media (e.g., RAM, hard disks, solid state memory) are widely used as information technology to model real-world entities and relationships. When real-world information about those entities and relationships added, the data structure may be referred to as a database. The defined structure within the data structure may impact system efficiency, query efficiency, query effectiveness, maintenance and update of information, and other properties. These properties may affect both users seeking information and contributing users that are adding, removing, maintaining, and/or otherwise contributing to information in the database. Some entities that can be modeled, for example, are businesses, a set of products and/or services they offer, employees or other personnel who work for or with the business, and the customers who at the business. However, certain entities and relationships may pose a significant challenge for modeling and electronic representation due to their complexity. Improperly modeled, structured, and stored electronic information can lead to inefficiencies for both users seeking information and users contributing to the database.

One relatively complex modeling challenge for a database may be healthcare related information. First, the relationship among entities is complex and can become quickly outdated. For example, relationships within the healthcare industry may be difficult to understand, such as when professionals may work at multiple clinics run by as separate businesses. Similarly, multiple users contributing to information in the database may be knowledgeable enough, but some may have more knowledge to describe (or authority to dictate) the real-world relationship. These relationships, modeled by references within the data structure, may be important for a user seeking information to understand context, affiliation between entities (including as may help evaluate reputation), and alternatives between and among healthcare solutions.

Second, users seeking information may find it difficult to navigate the taxonomies and/or terminologies pervasive in the healthcare industry. Such taxonomy and terminology may be helpful (or in some cases necessary) in defining the data structure that can accurately reflect the healthcare information and its intricacy. However, for many users seeking information, the terminology and taxonomy can be complex and difficult to understand or remember. For example, keyword searchers may not return satisfactory results due to the user's lack of information about their needs and/or their dearth of medical vocabulary on which natural keywords searches could otherwise attach within a database. When defining large databases with multiple contributing users the problem may compound over time as terminology in each specialty builds up in various parts of the database.

Attempting to educating oneself by wading into the medical taxonomy may be no less daunting for some users. Searchable databases with existing taxonomies may store information in hierarchical data structures that could lead the user into “dead ends” that may require them to go back and start over (e.g., a user being asked to find a service by first choosing specialty, sub-specialty, and then sub-sub-specialty, then discovering nothing seems to apply). By observing a user attempting both processes, it also be difficult to infer meaning or determine a user's needs.

When stored information lacks the structure to accurately model the real world, users contributing to information may add that information in ways that are difficult to maintain or for users seeking information to find. Users seeking information may not find adequate healthcare treatment and businesses may lose out on patients. When stored information lacks the structure to incorporate information that provides accessibility to users the users contributing to information (often representing businesses) may miss out on revenue. The seeking user may be unable to determine context or relationships, including affiliation or alternatives. The seeking users may have difficult identifying their needs, learning about their needs, and finding a solution for their needs. In some cases, they may even start their healthcare episode in the wrong starting place, wasting valuable weeks or months. As a result, users may remain untreated or ill and businesses may suffer lower patient satisfaction and declined revenue.

### SUMMARY

Disclosed are a method, a device, and/or a system of efficient determination of a data entity storing healthcare data through mapped entry and/or traversal of a semantic data structure.

In one embodiment, a method includes generating an entry criteria data for calculation of an entry point into one or more nodes of a semantic data structure. The entry criteria data includes a body part value and/or a body organ value. The entry criteria data also includes a general category value, a specific category value that is specific to the body part value and/or the body organ value, and/or a disease value.

The method receives a preference data of the user. The semantic data structure is queried to receive a first set of unique identifiers of a plurality of data entities each including a tag data matching the entry criteria data. The tag data matching the entry criteria data is mapped from a term value of a medical taxonomy through a tag reference to the tag data. The plurality of data entities include solution profiles that include a service profile, a professional profile, and/or a technology profile.

The method removes a first subset of the first set of unique identifiers based on a location data associated with the user stored in the user profile and/or the preference data of the user to result in a second set of unique identifiers. A card data for each data entity associated with the second set of unique identifiers is then transmitted to a client device of the user.

The method may receive a first profile request for a first solution profile including a unique identifier of the first solution profile, and may transmit to the client device of the user a profile data of the first solution profile.

One or more references of the first solution profile may be traversed within the semantic data structure to a set of solution profiles. The method may: (i) store a third set of unique identifiers comprising the set of solution profiles referenced through the one or more references of the first solution profile upon receiving the first profile request; (ii) remove a second subset of unique identifiers from the third set of unique identifiers based on the location data associated with the user stored in the user profile and the preference data of the user to result in a fourth set of unique identifiers; and (iii) transmit to the client device of the user a card data for each solution profile associated with the fourth set of unique identifiers. The user may therefore be to presented with semantically related data entities traversable by the user within the semantic data structure to increase a probability of an efficient connection between the solution profile that is applicable to the user.

The method may initiate a traversal criteria data initially comprising the entry criteria data. A tag data of the first solution profile may be extracted upon receiving the first profile request and the tag data of the first solution profile stored in the traversal criteria data. The third set of unique identifiers may include a second set of solutions profiles referencing the tag data of the first solution profile.

The method may determine that a stored reference within in the semantic data structure as an attribute of a second solution profile of the set of solution profiles is a unidirectional reference to a third solution profile. The stored reference may be determined to be prioritized based on a profile verification data, an entity type data, an entity size data, and/or a timestamp of the stored reference. The stored reference of the second solution profile may be an expressed reference. A unique identifier of the second solution profile may be included in the third set of unique identifiers. The client device of the user may transmit a card data of the third solution profile to increase a probability of modeling accuracy through expression of the stored reference as the expressed reference.

The method may also store a reference from the user profile to a unique identifier of the first solution profile to enable the user to re-query the first solution profile. A second profile request may be received for the solution profile comprising a unique identifier of the solution profile. To the client device of the user may be transmitted the card data of the first solution profile and/or the profile data of the first solution profile upon receiving the second profile request. Upon receiving the second profile request, one or more references of the first solution profile may be re-transmitted. A fifth set of unique identifiers may then be stored. A card data for each data entity associated with the fifth set of unique identifiers may be transmitted to the client device of the user to present re-calculated semantically related data entities traversable by the user.

The method may generate at a first time a first reference snapshot data of one or more references referencing a solution profile saved by the user. The first reference snapshot data may include a unique identifier one or more data entities referencing the solution profile. A second time a second reference snapshot data of one or more references referencing the solution profile saved by the user may be generated. The second reference snapshot data may include a unique identifier of one or more data entities referencing the solution profile. The first profile data at the first time and the first profile data at the second time may be determined to vary. A variation data may then be transmitted to the user.

The method may initiate a training session and extract a sixth set of unique identifiers of a set of solution profiles that have a set of data cards expanded by the user, a profile data requested by the user, and/or a set of stored references from the user profile one or more solution profiles. Receipt of an action request of the user associated with a fourth solution profile may be determined, and the unique identifier of the fourth solution profile extracted. The method may input as a first precursor path comprising the sixth set of unique identifiers as a first set of one or more inputs into an artificial neural network. The artificial neural network may include two or more processing nodes each including one or more weighted outputs. An applicable selection that may be the unique identifier of the solution profile and/or a tag data of the fourth solution profile may be input. The method may then adjust an output weight of one or more of the one or more weighted outputs to train the artificial neural network.

The method may extract a seventh set of unique identifiers of a set of solution profiles that have a different set of data cards expanded by the user, a different profile data requested by the user, and/or a different set of stored references from the user profile drawn to one or more solution profiles. As a second precursor path, the seventh set of unique identifiers as a second set of one or more inputs may be input into the artificial neural network. The method may then output from the artificial neural network a second tag data and/or the unique identifier of a fifth solution profile. The method may then query (i) the fifth solution profile with the unique identifier of the fifth solution profile and/or (ii) a sixth solution profile referencing the second tag data. A card data of the fifth solution profile and/or the sixth solution profile may be transmitted to the client device of the user.

A first reference in the first solution profile may be stored within the semantic data structure, the first reference drawn from the first solution profile to the second solution profile to assert a relation between the first solution profile and the second solution profile.

A second user controlling the second solution profile may be authenticated. A request may be received to delete the first reference within the semantic data structure, and the method may then compare a unique identifier of the first solution profile and a unique identifier of the second solution profile. A reference to an authority hierarchy data may be made, including determining the second solution profile is higher in the authority hierarchy data. The method may then delete the first reference in the first solution profile and/or designate the first reference as an unexpressed reference within the semantic data structure.

The method may include extracting a card data of the first solution profile, extracting a card data of the second solution profile, and generating a nested card data that incorporates at least a portion of the card data of the first solution profile into the card data of the second solution profile. The nested card data for the second solution profile may be transmitted to the client device of the user.

In addition, the method may read a relation status value from the first solution profile and transmit the relation status value within the card data of the first solution profile for display on the client device of the user to communicate an association strength within the semantic data structure of the first solution profile to the second solution profile. The tag data matching the entry criteria data including the body part value, the body organ value, the general category value, the specific category value, and/or the disease value, may be mapped to a term value of the medical taxonomy through the tag reference to the tag data.

The method may receive a third profile request for an expanded card, the third profile request including a unique identifier of a seventh solution profile. To the client device of the user may be transmitted an expanded card data for the seventh solution profile. It may be determined a first tag references a second tag data, and the second tag data may be added to the traversal criteria data. The method may remove the preference data and/or expanding a location preference of the user profile. The entry criteria data may further include and a property data of the user.

In another embodiment, a computer readable medium stores a data structure for efficient determination of a medical solution applicable to a user. The compute readable medium includes a first data entity that includes a unique identifier of the first data entity and a content data of the first data entity specifying a name of a first solution profile and/or a description data describing the first solution profile. The first data entity further includes an action function of the first data entity comprising a set of computer readable instructions and/or a reference to the set of computer readable instructions that when executed, initiate an action associated with the first solution profile upon receipt of an action request generated by a client device.

The data entity further includes a first reference attribute of the first data entity storing an identifier of a second data entity to define a first reference that is unbound by hierarchical constraint within the data structure. The data entity includes a tag data and/or a tag reference to the tag data. The tag data includes a first term value mapped to a second term value, where the second term value is selected from a medical taxonomy.

In yet another embodiment, a system for efficient database query includes a database server, a coordination server, and a network communicatively coupling the coordination server and the database server. The database server includes a processor of the database server, a memory of the database server, and a semantic data structure. The semantic data structure includes a plurality of solution profiles as nodes of the semantic data structure. Each node is coupled to one another through a chain of one or more references unbound by hierarchical constraint.

The coordination server includes a processor of the coordination server, a memory of the coordination server, a request agent, an entry subroutine, a filter subroutine, and a card extraction module. The request agent includes computer readable instructions that when executed receive from a client device of a user an entry criteria data usable to determine an entry node of the semantic data structure. The entry criteria data includes a body part value and/or a body organ value. The entry subroutine includes computer readable instructions that when executed: (i) determine an entry point into one or more nodes of the semantic data structure, and (ii) query the semantic data structure to receive a first set of unique identifiers of the plurality of solution profiles each includes a tag data matching the entry criteria data. The tag data is mapped to a term value of a medical taxonomy through a tag reference.

The filter subroutine comprising computer readable instructions that when executed remove a first subset of the first set of unique identifiers based on a location data associated with the user stored in a user profile of the user and/or a preference data of the user, to result in a second set of unique identifiers. The card extraction module includes computer readable instructions that when executed extract a card data for each data entity associated with the second set of unique identifiers and transmit to a client device of the user the card data for each data entity associated with the second set of unique identifiers. The system may further include a machine learning server and the client device.

### BRIEF DESCRIPTION OF THE DRAWINGS

The embodiments of this disclosure are illustrated by way of example and not limitation in the figures of the accompanying drawings, in which like references indicate similar elements and in which:

FIG. 1A is a network for efficient determination of a data entity storing healthcare information in a solution profile stored in a semantic data structure, the network comprising a database server storing the semantic data structure, a coordination server comprising a traversal engine for traversing the semantic network to extract information and assist in leading the user to applicable solution profiles, and a machine learning server assessing a traversal data and generating an output that may be used predict applicable selections for the user, a one or more client devices of users seeking information and/or contributing to the semantic data structure, according to one or more embodiments.

FIG. 1B is a data entity data structure illustrating three data entities that may be used for modeling real-world entities, including illustration of an entity reference having associated a reference modification data that changes a meaning of an entity reference, an entity having an associated designation data usable to describe a relation status (e.g., to help evaluate its certainty and/or strength), a derivative reference that can derive information for one entity from another entity, and a tag reference to a tag data mapping a first term value (e.g., a common term) to a second term value (e.g., a medical term from a medical taxonomy), according to one or more embodiments.

FIG. 1C is a data structure view illustrating five solutions profiles instantiated from a data entity of FIG. 1B and that may be nodes within the semantic data structure (a professional profile, a business profile, a technology and/or product profile, a service profile, and/or an educational material profile), each solution profile including a unique identifier, a content data, an action function, a tag reference, and a set of referential attributes that can reference one or more of the other solution profiles unbound by hierarchical constraint to enable free-flowing and natural query of the user, according to one or more embodiments.

FIG. 1D is another data structure view illustrates additional example data entities and relations that may be defined within the semantic data structure, including references to and/or from each of the data entities of FIG. 1C, including a specialty profile, a location profile, a manufacture profile, and/or vendor profile, according to one or more embodiments.

FIG. 1E is a data structure reference view illustrating additional reference types between data entities capable of increased accuracy in modeling relationships, including unexpressed references which may be hidden from a user upon query and expressed references that may be expressed to the user upon query, for example related to authority of one or more of the data entities relative to another and/or rules of priority which may be applied on or before query, to assist in complex modeling of relationships (for example within the healthcare industry), according to one or more embodiments.

FIG. 2 illustrates the database server of FIG. 1, including a database management system, a mapping database comprising tag data with a first term value mapped to a second term value of a medical taxonomy, and an entity database storing the semantic data structure, according to one or more embodiments.

FIG. 3 illustrates the coordination server of FIG. 1, including a request agent for receiving an entry criteria data usable to enter the semantic network at a node of initial relevance and/or for receiving a profile request data for specific data entity, a traversal engine to traverse the semantic data structure through feedback with the user to guide the user to an applicable solution, a user profile of a user seeking information and/or a user contributing to the semantic database, and an action response agent to receive and carry out an action of a solution profile upon the request of the user, according to one or more embodiments.

FIG. 4 illustrates a machine learning server comprising an artificial neural network, a training routine for training the artificial neural network with a first precursor path generated from a traversal data, a predication routine for using the artificial neural network to generate an applicable selection from a precursor path, and an example of an artificial neural network comprising, according to one or more embodiments.

FIG. 5 illustrates the client device of FIG. 1, including a request generation module for generation of an entry criteria data and/or a profile request data, and a display application for displaying tiered levels of data cards generated from card data extracted from entry and/or traversal of the semantic data structure, according to one or more embodiments.

FIG. 6 is a semantic data structure entry view illustrating assembly of the entry criteria data received by the request agent of FIG. 3 for initial entry into one or more nodes of the semantic data structure, including possible selection of one or more pieces of data matchable to tag data, for example a body part value (e.g., “head”), a body organ value (e.g., “immune system,”), a general category value (e.g., “surgery”), a specific category value that is specific to the body part value and/or the body organ value (e.g., “auto-immune”), and/or a user property (e.g., “female”), according to one or more embodiments.

FIG. 7 illustrates an interface view as may be observed by a user as the result of the entry and/or traversal of the semantic data structure, including a tiered presentation of information for rapid learning and query efficiency utilizing expandable data cards for continual compact display of semantic relations, according to one or more embodiments.

FIG. 8 is a data entity generation process flow illustrating a process for generating the data entity of FIG. 1B, a solution profile of FIG. 1C, and/or other data entities, according to one or more embodiments.

FIG. 9 is a reference authority process flow illustrating a process for determining authority for a first data entity to delete a reference and/or designate the reference to be unexpressed within the semantic data structure to enable increased accuracy of the database modeling, according to one or more embodiments.

FIG. 10 is a semantic data structure entry process flow further illustrating a process for entry into one or more nodes of the semantic data structure, according to one or more embodiments.

FIG. 11 is a semantic traversal process flow illustrating a process for traversing references that may be unbound by hierarchical constraint within the semantic data structure, including possible filtering of initial results with a preference data and/or expansion through an expansion process before return of a result data, to assist the user in finding a relevant solution profile, according to one or more embodiments.

FIG. 12 is a reference prioritization process flow illustrating a process for resolving reference priority in order to express or unexpress a reference to assist in complex modeling of relations between data entities, for example within the healthcare industry, according to one or more embodiments.

FIG. 13 is a reference save and compare process flow illustrating a user profile saving a data card of a solution profile, optionally along with its incoming and/or outgoing references at a first time, and possibly later comparing incoming and/or outgoing references at a later time such that the user may comprehend the changes in relation among other entities in the semantic data structure, according to one or more embodiments.

FIG. 14 is a machine learning training process flow for training an artificial neural network with a precursor path and an applicable solution to output a unique identifier of a solution profile and/or a unique identifier of a data tag, according to one or more embodiments.

FIG. 15 is a machine learning prediction process flow for utilizing the artificial neural network of FIG. 14 to output the unique identifier of the solution profile and/or the unique identifier of the data tag as the applicable solution upon input of a different precursor path, according to one or more embodiments.

FIG. 16 is a card nesting process flow illustrating a process for incorporating a data card of a first data entity into a data card of a second data entity, for example to flexibly generate combinations of data that may otherwise be separately modeled, according to one or more embodiments.

FIG. 17 illustrates an example user interface view of a workflow available to the user, for example on the client device, for selecting body part value, a body organ value, a disease value, and/or a category value usable for determining one or more initial entry nodes into the semantic data structure, according to one or more embodiments.

FIG. 18 illustrates another example user interface view illustrating a set of data cards generated from card data extracted from a set of solution profiles following initial entry into one or more nodes of the semantic data structure as shown in FIG. 17, according to one or more embodiments.

FIG. 19 illustrates yet another example user interface view illustrating a profile data of a solution profile as a main focus selected from one of the data cards of FIG. 18, additionally illustrating three data cards of solution profiles referenced by and/or drawing references to the solution profile that is the main focus, according to one or more embodiments.

FIG. 20 illustrates yet another example of a user interface view illustrating a profile data of a business profile selected from a data card of FIG. 19 as a main focus, with three data cards of solution profiles at least one of referenced by and/or drawing references to the solution profile that is the main focus presented as ‘dots’ selectable to open an expanded data card, the dots positioned on an image associated with the solution profile that is the main focus to permit accessible visual transition to assist the user in traversing the semantic data structure, according to one or more embodiments.

Other features of the present embodiments will be apparent from the accompanying drawings and from the detailed description that follows.

### DETAILED DESCRIPTION

Disclosed are a method, a device, and/or system of efficient determination of a data entity storing healthcare data through mapped entry and/or traversal of a semantic data structure. Although the present embodiments have been described with reference to specific example embodiments, it will be evident that various modifications and changes may be made to these embodiments without departing from the broader spirit and scope of the various embodiments.

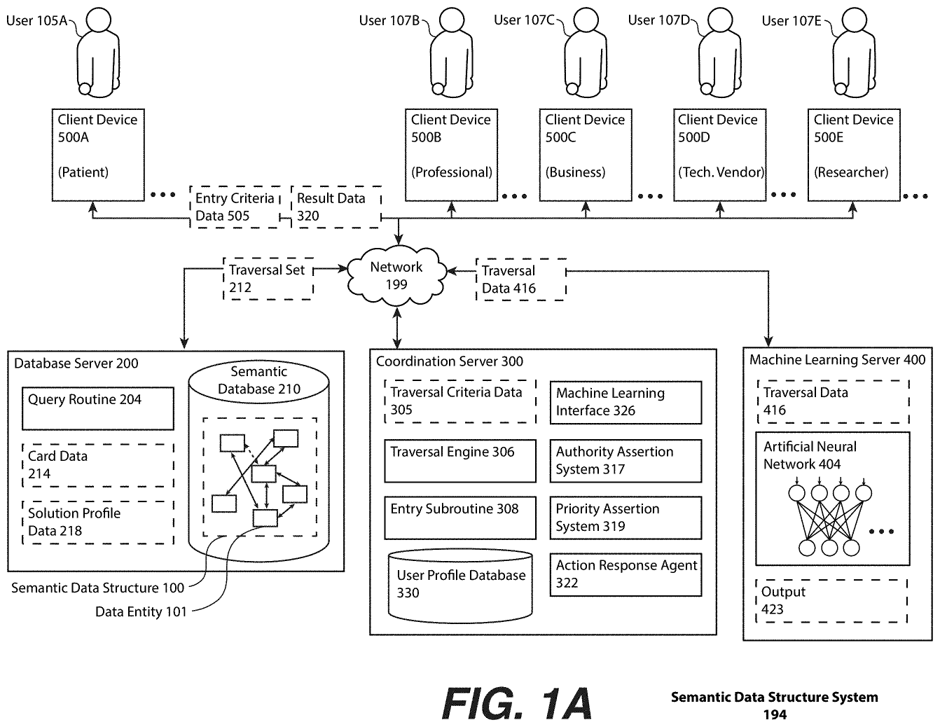

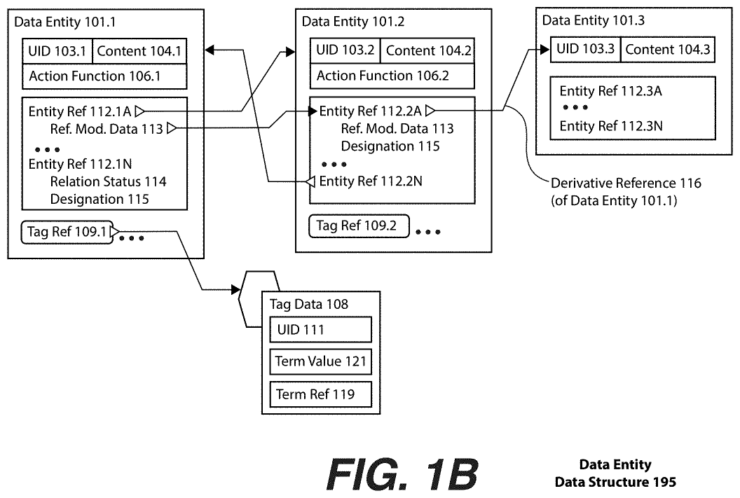

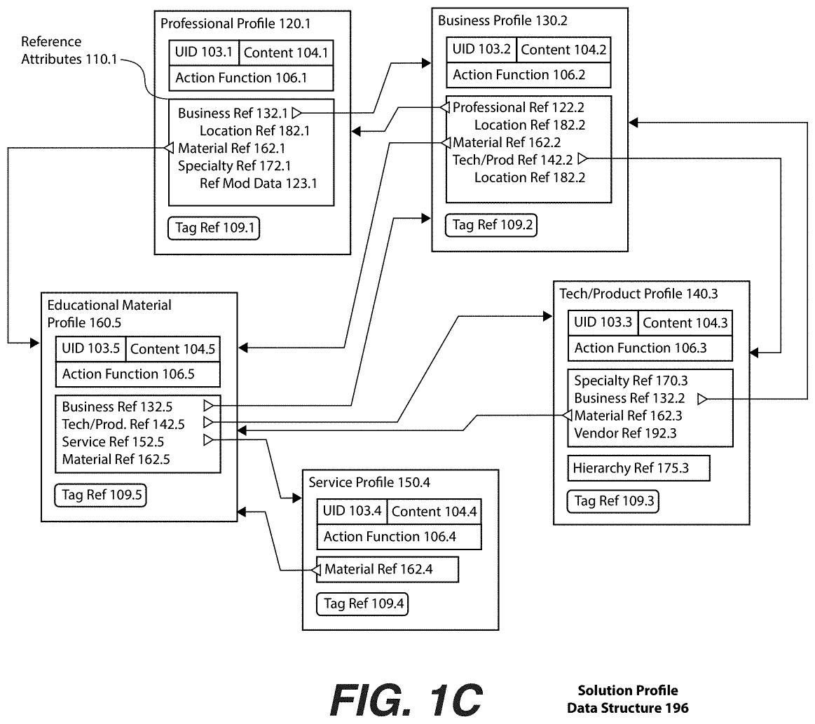

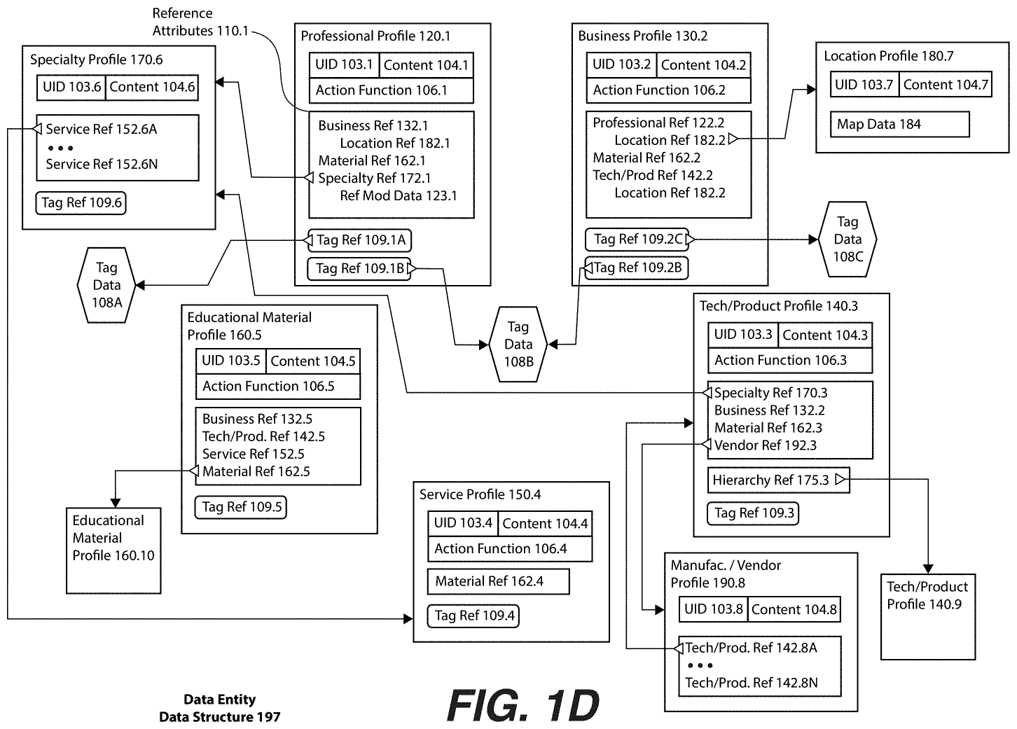

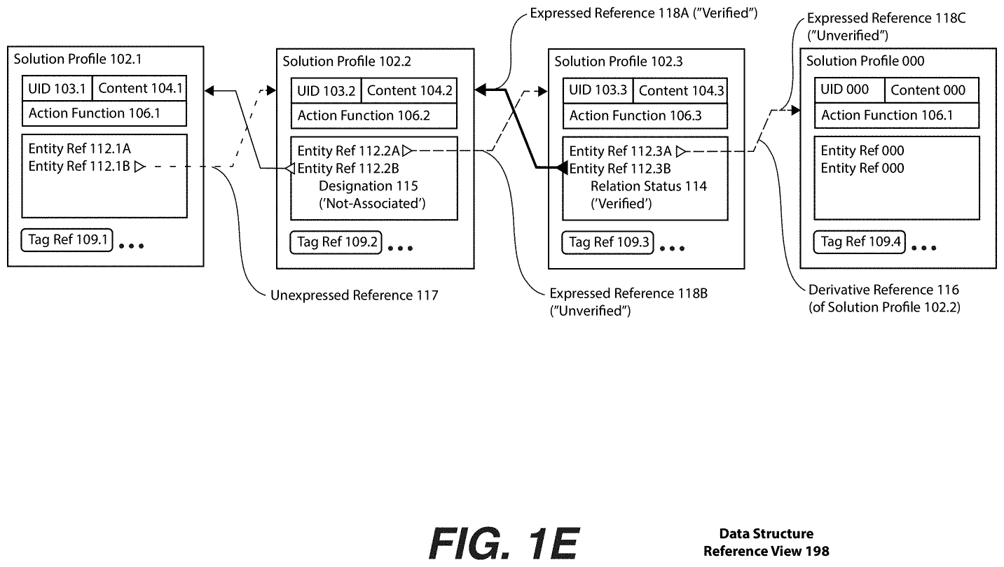

FIG. 1A illustrates a semantic data structure system 194 comprising a network communicatively coupling a database server, a coordination server, a machine learning server, and one or more client devices, according to one or more embodiments.

A user 105A (e.g., a consumer, a patient, a professional learning about an area outside his or her expertise, etc.) may be searching for a health solution. The user 105A may have a general sense of need (e.g., a desire for better back health) and/or a specific objective (e.g., evaluating alternatives to LASIK surgery). The user 105A utilizes a client device 500A (e.g., a smartphone, a laptop computer) to generate an entry criteria data 505. The user 105A may be registered (e.g., an authenticated user having a user profile 339 stored in a user profile database 330) and/or may be unregistered.

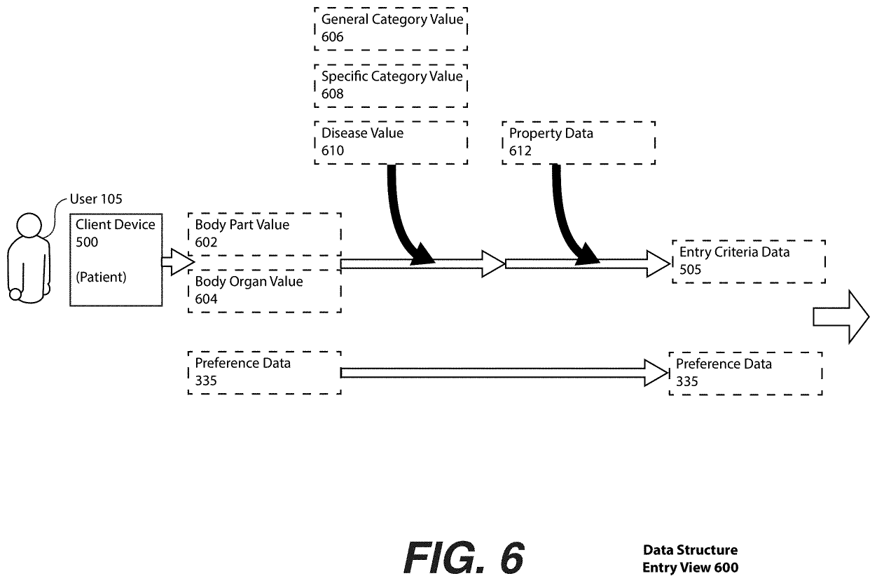

The entry criteria data 505 may include a relatively simple set of one or more terms and/or categories with which the user 105A may be familiar, for example the names of body parts, body organs, common disease or condition names, and general healthcare categories and/or specific healthcare categories. The entry criteria data 505 may be generated by a workflow or menu to build an initial set of data usable to find an entry point into the semantic data structure 100. An example of the generation of the entry criteria data 505 is shown and described in detail in conjunction with FIG. 6, and other examples are provided throughout the present embodiments. The entry criteria data 505 may be transmitted through the network 199 to the coordination server 300. The network 199, for example, may be a local area network (LAN), a wide area network (WAN), a virtual private network (VPN), and/or the internet.

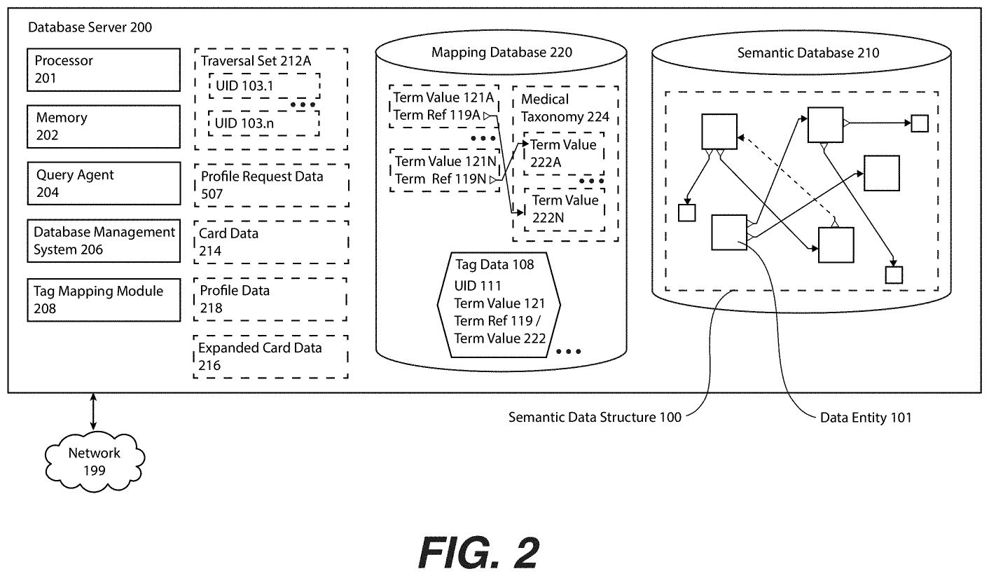

The coordination server 300 receives the entry criteria data 505. A traversal engine 306 comprises computer instructions for entering and traversing references of the semantic data structure 100 (e.g., an entity reference 112 of FIG. 1B). An entry subroutine 308 may include computer instructions that determine a mapping between the entry criteria data 505 and one or more terms in a medical taxonomy (e.g., the medical taxonomy 224 of FIG. 2) that may then be used to search the semantic database 240 for an entry point. In one or more other embodiments, a matching may occur through one or more terms of the entry criteria data 505 matched to one or more tag data 108 associated with data entities 101 within the semantic data structure 100, as shown and described in conjunction with the embodiments of FIG. 1B, FIG. 1D, and FIG. 2.

The semantic data structure 100 and the processes which operate on it may be, in one or more embodiments, a system that (i) stores entities; (ii) defines relations between entities; (iii) may derive meaning and derived information for a given entity from one or more referenced entities; and (iv) defines structures enabling control over both the derivate information, including enabling control over the expression of references between data entities. The system of control may further include systems of priority and/or authority that express or unexpress references (and therefore modeled relations) depending on factors such as how likely one entity is to have the authority to make a decision regarding a relation, how likely one entity is to know the relation between two entities, and similar considerations.

The coordination server 300 may call the query agent 204 of the database server 200. The database server 200 includes a semantic data structure 100 that comprises a plurality of data entities 101 interconnected through references (e.g., as shown and described in conjunction with the embodiment of FIG. 1B through FIG. 1E). Along with specific healthcare information stored in the data entities 101, the semantic data structure 100 may be referred to as the semantic database 210.

Following determination of one or more entry points into the semantic data structure 100, the query agent 204 may return a traversal set 212 identifying relevant data entities 101, for example as identified by unique identifiers (e.g., a unique identifier 103 of FIG. 1B). A subset of the traversal set 212 may be selected, for example by applying a filter to reduce the number of results and/or applying a filter to account for user preferences (e.g., location, gender of a professional, price of a service, accepted insurance, etc.). Following any changes (e.g., filtering and/or expansion), the query agent 204 may query the semantic database 240 for data from each data entity 101 in the subset. For example, entry point(s) may result in query for data of data entities 101 instantiated as solution profiles 102, including four instances of each of a professional profile 120, a business profile 130, a technology/product profile 140 (shown as the “tech/product profile 140”), a service profile 150, and an educational material profile 160 (e.g., as each may be shown and described in conjunction with FIG. 1C).

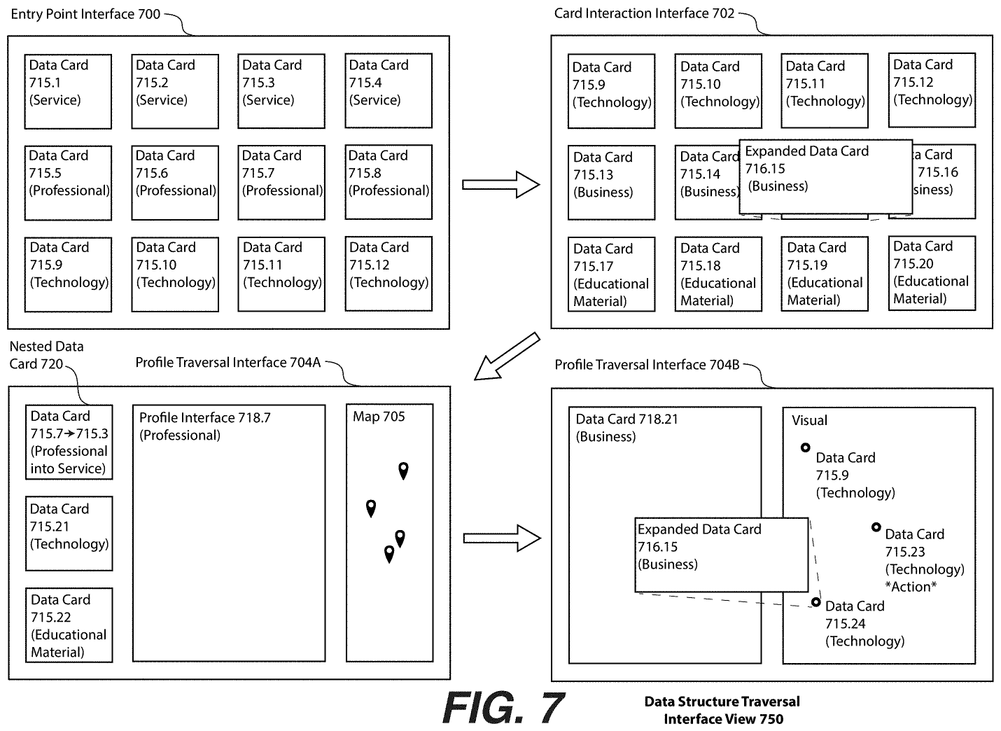

The query may extract a subset of data from each solution profile 102, referred to as the card data 214. The card data 214 may allow for generation of a succinct “data card” on a user interface, including an optional expandable view (e.g., an expanded data card) to in which the data card is “unfolded” (e.g., the data card 715 and the expanded data card 716 as shown and described in conjunction with FIG. 7). A result data 320 that includes the set of card data 214 may be returned as the result data 320 to the client device 500A of the user 105A. A software application running on the client device 500A may then convert the card data 214 into a visual representation on a user interface. The user 105A may be able to quickly review the resulting data cards 715, for example rapidly gathering additional information by expanding one or more data cards 715 of interest and, if the user 105A believes they are heading toward a solution, may request the full set and/or larger dataset of the solution profile 102, e.g., the profile data 218. The user 105A may therefore have: (i) found an entry point into the semantic data structure 100, and (ii) selected an initial branching point for initiating traversal of the semantic data structure 100. In one or more embodiments, the user 105A may now have a multifaceted approach that may not presume a specific need, may not force the user 105A into what may be a rigid taxonomy, and may not commit the user 105A to a pathway that requires significant energy to withdraw from if later determined by the user 105A to be inapplicable.

The user 105A may identify a solution profile 102 of interest through its user interface representation. The client device 500A may generate a profile request data 507 (not shown) for retrieval of the profile data 218 of the solution profile 102, for example from which the card data 214 was extracted as may have been viewed by the user 105A as the data card 715. The traversal engine 306 may include computer instructions which call the database server 200 to query the solution profile 102 for the profile data 218. The call may simultaneously request and/or initiate traversal of one or more references of the solution profile 102 that may form a branch point of the semantic data structure 100. The traversal may be a single traversal, for example moving from a professional profile 120 to an educational material profile 160 (e.g., authored or co-authored by a professional such as the user 107B controlling the professional profile 120 and possibly the educational material profile 160). The traversal may also be multiple traversals, for example following a first reference from a first professional profile 120A to a business profile 130, then along a second reference from the business profile 130 to a second professional profile 120B. The data entities 101 and/or solution profiles 102 found during traversal may then be assessed for priority and/or authority, filtered, expanded, and/or otherwise adjusted. A second instance of the result data 230 may then be returned including the profile data 218 of the solution profile 102 that may be primarily requested. The remaining subset of data entities 101 may then each have a card data 214 extracted for return within a second instance of the result data 320.

Upon receiving the second instance of the result data 320, the software application running on the client device 500A may convert the card data 214 into another visual representation on the user interface. In this instance, the profile data 218 of the solution profile 102 may include more data than the card data 214. In one or more embodiments, the user interface may therefore focus or emphasize the profile data 218 of the solution profile 102 while also including space for display of the “semantic relations” of the solution profile 102 as data cards 715 corresponding to each card data 214 returned in the second instance of the result data 320.

As the user 105A traverses the semantic data structure 100, a traversal criteria data 305 may be generated which may modify and/or add to the entry criteria data 505. For example, it may be identified that the user 105A is actually interested in a different body part, disease, and/or category then they initially thought, allowing for additional matches and/or filtering during continued traversal and possibly continuously changing the nature of the result data 320 generated to keep track of the user 105's changing and/or evolving interests and goals.

The user 105A may repeat the process of selection, and/or may take one or more actions that lead to a definite outcome and/or indicate that the user 105A has arrived at a solution profile 102 applicable to the user 105A. For example, the user 105A may select on a user interface of the client device 500A an “action button” linked to an action function 106 referenced, specified, and/or defined in computer readable instructions within the solution profile 102 (e.g., the action function 106 of FIG. 1C). For example, the action may appear to the user 105A as “schedule an appointment,” “take a tour,” or “request a brochure.” Triggering of the action function 106 of the solution profile 102 may result in a concrete and measurable step taken by the user 105A demonstrating the applicability of that solution profile 102 to the user 105A. The client device 500A generates an action request data 509 which is received by the action response agent 322 which may comprise computing instructions that execute the action, including possibly gathering additional information from the user 105A, coordinating with third-party APIs, and other coordination processes.

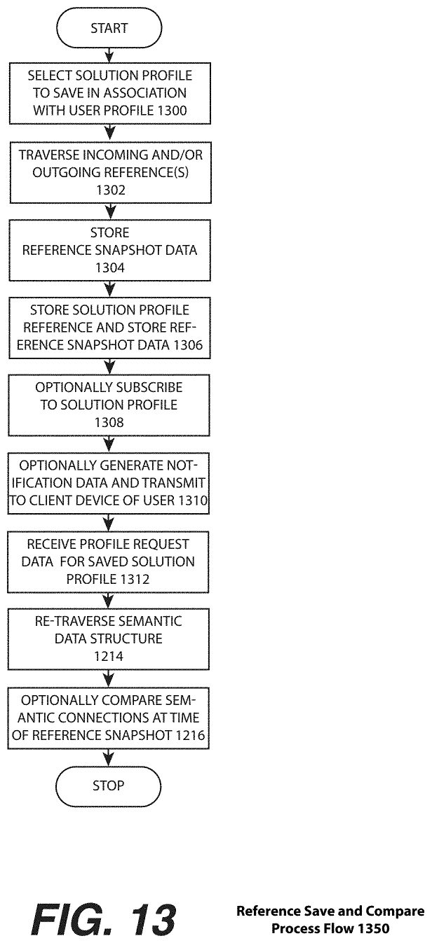

The user 105A may continue the process exploring information extracted from data entities 101, possibly initiating multiple actions throughout the course of a session exploration. The user 105A may also carry out a number of useful supportive and/or primary tasks, such as saving data cards 715 and/or later comparing semantic references of the a data entity 101 associated with a saved instance of the data card 715 (e.g., as shown and described in conjunction with the embodiment of FIG. 13). This may allow the user 105 to determine how relations have changed, possibly helping the user 105 to asses context, affiliation, and re-assess reputational associations. Through the process of traversal, card expansion, action triggering, and card saving, a machine learning interface 326 may generate and send data from the interactions of the user 105A to a machine learning server before, during, and/or after the traversal process to train the artificial neural network 404 and/or to assist in determining relevant selections.

The machine learning server 400 comprises an artificial neural network that receives inputs and “recognizes” a pattern of interactions and/or events to generate a prediction. In one or more embodiments, the machine learning server 400 receives a traversal data 416 that includes information about the user 105A's interactions with the semantic database 240. For example, the traversal data 416 may include the entry criteria data 505, the profile request data 507, the traversal set 212, the result data 230, and/or other data such as related to UI interactions such as data card 715 expansions, attention tracking (e.g., mouseovers and/or eye tracking), and other interactions. The traversal data 416 along with an applicable selection of a data entity 101 may be used to train the artificial neural network 404 (e.g., to recognize that when the user 105A traverses a given path, they are likely to select a certain instance of a solution profile 102). Once sufficiently trained, the machine learning server 400 may then accept traversal data 416 and generate an output 423 that is a predicated applicable selection for the user 105A and/or other useful data, such as an addition and/or modification to the traversal criteria data 305 (e.g., adding a tag data 108). As a result, the user 105A may be able to efficiently connect to one or more solution profiles 102 that may be applicable to the user 105A, including which may solve a major healthcare challenge and/or meet a healthcare objective of the user 105A.

An initial description of the semantic data structure and its setup is now provided. A semantic data structure 100 including a plurality of data entities 101 may be stored on a memory of a server. The data entity 101 may be instantiated into electronic profiles modeling certain real-world entities providing one or more aspects of a healthcare solution. For example, the data entity 101 may be instantiated into a solution profile 102 (not shown in the embodiment of FIG. 1A) for healthcare professionals (e.g., doctors, nurse practitioners, physical therapists, surgeons, etc.), healthcare businesses (e.g., clinics, hospitals, doctors' offices), technology and/or products (e.g., a type of medical device, a type of medical equipment such as an MRI or laser eye surgery machine, a pharmaceutical, a type of health supplement), and educational material (e.g., an editorial, an encyclopedia entry, a research paper, a medical review article, a study, a journal article, etc.), As shown and described throughout the present embodiments, each of the data entities 101 may have one or more references stored within the data entity 101 drawn to one or more other data entities 101 unbound by hierarchical requirements within the semantic data structure 100 to create a rich set of interconnections that may be capable of accurately describing (and maintaining in real time) real-world complex relationships.

A set of users 105 may contribute to the semantic database 210, for example the user 107B through the user 107E. Each of the users 105 may, for example, be able to define new data entities 101, claim control over existing data entities 101, and add and/or remove data from data entities 101, including the definition of one or more references drawn to other data entities 101 to assert various relations between data entities 101. As shown and described throughout the present embodiments, certain references may be authorized for expression or prioritized such that they are expressed (e.g., resulting in certain presented relations shown upon query) and/or certain other references may be unauthorized (and therefore may be unexpressed in one or more queries). The authority assertion system 319 and the priority assertion system 313 are shown and described in conjunction with one or more of the embodiments, below. In one or more embodiments, a user may have multiple roles, for example a user 107 having authority to own and/or control data entities 101 may also be able to act as a user 105 who enters and/or traverses the semantic data structure 100 (although the reverse not always tree—there may be many users 105 with no ownership and/or control of data entities 101).

A numbering convention used in one or more of the embodiments, including FIG. 1B through FIG. 1E, will first be described. In one or more of the present embodiments, a number leading a decimal point designates a type of element (e.g., the data entity 101), a number trailing the decimal point may represent an instance of the element (e.g., the data entity 101.1 is a first instance of the data entity 101) and/or a different element associated with the element (e.g., the content data 104.1 of the data entity 101.1 is a content data 104 contained by the data entity 101.1). A letter trailing the decimal point may designation an instance of the element associated with the element (e.g., the entity reference 112.1A and the entity reference 112.1B may be two entity references 112 of the data entity 101.1). Throughout the present embodiments and in the drawings, “ref” is used synonymously with the term “reference”. For example, the entity reference 112 may be shown in the drawings as the “entity ref 112.”

FIG. 1B illustrates a data entity data structure view 195, illustrating several possible aspects of the semantic data structure 100, according to one or more embodiments. First, a general description of a data entity 101 and some of its possible components are provided, followed by a further description of the data entity 101.1 through the data entity 101.3 of FIG. 1B.

A data entity 101 is a set of machine-readable information stored as bits in machine readable media (e.g., random access memory (RAM), a storage disk, etc.), for example electronically and/or magnetically. The data entity 101 includes a unique identifier 103 that allows for the data entity 101 to be individually addressed within the semantic data structure 100, for example an alphanumeric name, a randomly generated 32-character alphanumeric string (e.g., a globally unique identifier or GUID) and other identifiers as may be known in the art (throughout the present embodiments including in the drawings, the unique identifier 103 and/or other unique identifiers may be referred to as a “UID” for brevity, e.g., the “UID 103”). The data entity 101 also includes a set of one or more reference attributes 110. Each reference attribute may store a reference to another data entity 101, for example by storing an identifier of the other data entity 101. The unique identifier 103 may be stored as a value of the reference attribute.

The data entity 101 may include a content data 104. The content data 104 may include a number of stored attributes with associated values describing and/or defining properties of the data entity 101. For example, the content data 104 may specify a name of the data entity 101 and/or a description of the data entity 101. In a specific example, the data entity 101 may be instantiated as a professional profile 120 (e.g., as shown in FIG. 1C), where the content data 104 includes the name of the professional, a set of credentials held by the professional, a biography of the professional, and other information. In another example, the data entity 101 may be a specialty profile 170 (e.g., as shown in FIG. 1D), where the content data 104 stores text describing the specialty, common synonyms, and other specialties to which it may relate. Certain data which might be thought of as describing the data entity 101 may be gathered through reference and/or a derivative reference (e.g., a business where the professional works, and a specific location where they are sometimes present when working).

The data entity 101 may also include one or more instances of an action function 106, which may specify, reference, and/or comprise computer readable instructions for initiating an action with request to the data entity 101. For example, the data entity 101 may include a set of computer executable instructions and/or a reference to a set of computer executable instructions that, upon receipt of an action request generated by the user 105A (e.g., receipt of the action request data 509 as generated on a client device 500A) initiates an action associated with the first solution profile 500. For example, where the data entity 101 is a solution profile 102, as further described below, the action function 106 may form a part of the “solution” represented and/or modeled by the solution profile 102. Triggering the action function 106 may also indicate that the user profile 339 has been connected to a data entity 101 (e.g., a solution profile 102) that is applicable to the user 105, whether meeting an objective, or even creating a new pivotal branching within the semantic network that may correspond to new insight and/or new objectives of the user 105.

The data entity 101 may also store one or more instances of a tag data 108 and/or one or more instances of a tag reference 109 to the one or more instances of the tag data 108. The tag data 108 may include data that maps one or more term values to one or more other term values. The tag data 108 may serve a number of purposes within the semantic data structure 100, including (i) a closest related term to a health solution, or (ii) a disease associated with the health solution, and (iii) a manner in which a health solution is performed (e.g., 24 hour service, same day service). In one or more embodiments, a first term value is a common word (e.g., “stomach”) that is mapped to a second term value from a medical taxonomy (e.g., “gastroenterology”). The mapping may be static and/or dynamic, as further described in conjunction with the embodiment of FIG. 2, below.

The set of reference attributes 110 may be one or more reference attributes that along with a value for each of the one or more reference attributes define one or more references to other data entities 101. Specifically, the entity reference 112 which may be the reference attribute and associated value may be a reference to a different data entity 101, where the value of the entity reference 112 is the unique identifier 103 of the different data entity 101. Depending on the type of data entity 101 referenced, different meaning of a relation may be explicitly defined and/or implied. For example, where the data entity 101.1 instantiated as a professional profile 120.1 having an entity reference 112.1A (e.g., instantiated as the business ref 132.1 of FIG. 1C) to the data entity 101.2 is instantiated as a business profile 130.2, the entity reference 112.1A may designate a clinic where a professional associated with the professional profile 120.1 offers services.

The set of reference attributes 110 may also include data which modifies a reference attribute, for example to change the explicit or implied meaning of a relation between data entities 101. A reference modification data 113 stored in association with the entity reference 112 may modify the meaning and/or filter content referenced from the data entity 101.2. This may provide some control to a controlling user 107A of the data entity 101.1 without disrupting a controlling user 107B of the data entity 101.2 from which the data derives. In one or more embodiments, the reference modification data 113 associated with the reference attribute of the data entity 101.1 stores data for, upon traversal of the entity reference 112.1A: (i) modification of the content data 104.2 of the data entity 101.2 and/or (ii) termination of continuing traversal of the reference 101.2. For example, where the data entity 101.1 is instantiated as the professional profile 120.1, the reference modification data 113 may specify a certain condition on which the entity reference 112.1A should not be traversed as part of the user 105A moving through the semantic data structure 100, e.g., a professional operates seasonally in an Alaskan clinic (e.g., represented by the data entity 101 instantiated as the business profile 130) and therefore only expresses their relation to that clinic four months out of the year. In another example, the reference modification data 113 may specify that certain other information that may otherwise be extracted and presented through query of the data entity 101 should be excluded (or, conversely, that additional information should be included). For example, when the user 105A requests full information of the professional profile 120.1, a number of locations where the associated professional may work may be presented on a map shown to the user 105A (e.g., the map 705 of FIG. 7). Each such location may be extracted by first determining the businesses associated with the professional (e.g., an entity reference 112.1 to a business profile 130.2), and then further traversing references of the business specifying their locations (e.g., an entity reference 112.2 to location profile 180.3). By default, all such locations may be extracted and presented in association with the data entity 101.1. In such case, the locations of the data entity 101.1 may be referred to as a derivative reference 116. However, the reference modification data 113 may override one or more of these imported and/or attributed pieces of information and/or references. For example, the reference modification data 113 may specify that the professional does not work at one of the referenced locations and such locations will be deleted from any profile data 218 such that it may not be displayed to the user 105. However, the referential use of such information may, upon closure of the business, automatically terminate the derivative reference 116, probably increase accuracy of modeling without action of the professional. In another example, the reference modification data 113 may be used to specify a referenced data entity 101.2 is not affiliated with the data entity 101.1, which may be a factor for determining authority and/or priority of references and their expression and/or traversal within the semantic data structure 100, as further shown and described in conjunction with the embodiment of FIG. 1E.

An entity reference 112 may further include an associated instance of a relation status 114. The relation status 114 may include data specifying whether a user 107 owning and/or controlling the data entity 101 has explicitly verified the entity reference 112 that may therefore verify the relation represented by the entity reference 112. Although not shown, an instance of the relation status 114 applying to the entire data entity 101 may also be applied and/or imputed to one or more reference attributes 110. For example, the data entity 101 may have a relation status 114 of “verified”, meaning a user 107 has claimed the ownership and/or control of the data entity 101. In one or more other embodiments, the data entity 101 and each entity reference 112 may have a distinct relation status 114. Similarly, there may also be levels of verification (e.g., stored in the profile verification data). For example, where the data entity 101 is verified, each entity reference 112 may be transitioned from an “unverified” status to a “partially verified” status for all entity references 112 of the reference attributes 110. Once a relation represented by a specific instance of an entity reference 112 is reviewed, the relation status 114 may the be updated to a “verified” status for that relation. The relation status 114 may be included in the profile data 218 of the solution profile 102 and may be presented to the user 105A to help indicate a likelihood of a relation between two data entities 101. The relation status may also be used in determining priority of entity references 112, and/or utilized in ranking results on a user interface for presentation to the user 105A (e.g., by the ranking subroutine 238).

While many entity references 112 may be stored in the set of reference attributes 110, not all may remain active and/or “expressed” at a given time, for a given query, and/or within a given context. As used herein, a stored reference is an entity reference 112 that is stored, and which may be either expressed or unexpressed in certain circumstances. A designation data 115 includes data which may be used to provide a designation to the entity reference 112 as to whether the entity reference is to be expressed and/or unexpressed during traversal of the semantic data structure 100. For example, the designation data 115 may represent a declaration that an entity reference 112 to which it is applied should not be expressed (e.g., an unexpressed reference 117 as shown and described in conjunction with FIG. 1E) or should be explicitly expressed (e.g., the expressed reference 118 as shown and described in conjunction with FIG. 1E). It should be noted that expression of a stored reference may also be determined in association with reference priority determinations, as shown and described further below.

A description of the data entity 101.1 through the data entity 101.3 of FIG. 1B is now provided. The data entity 101.1 includes an entity reference 112.1A to the data entity 101.2. The data entity 101.2 includes an entity reference 112.2A to the data entity 101.3. When data is extracted from the data entity 101.1, derivative data may also be extracted from the data entity 101.2 and/or the data entity 101.3 (e.g., the entity reference 112.2A is a derivative reference 116 of the data entity 101.1). The reference modification data 113 of the entity reference 112.1A may modify the data otherwise brought in through references to the data entity 101.2 and/or through the derivative reference 116, for example excluding certain information that would otherwise be extracted and/or included with the data entity 101.1 upon query.

The data entity 101.2 includes the entity reference 112.2N drawn to the data entity 101.1. Where the data entity 101.1 references the data entity 101.2, and the data entity 101.2 references the data entity 101.1, a “two way” reference may be defined which may strengthen the implied relation. In other cases, the two-way reference may weaking the relation, for example if a user 107 owning and/or controlling the data entity 101.1 applies a designation (e.g., from a designation data 115) to the entity reference 112.2N (not shown) specifying the data entity 101.1 is not affiliated with the data entity 101.2. References implying conflicting relations may lead to the two or more responsible references being expressed, unexpressed, added, or deleted based on authority and/or priority, as described below.

FIG. 1C is a solution profile data structure 196, according to one or more embodiments. Each of the solution profiles 102 of FIG. 1C may be an instantiation of the data entity 101 of FIG. 1B. In one or more embodiments, each of the solution profiles 102 of FIG. 1C (e.g., the professional profile 120, the business profile 130, the technology/product profile 140, the service profile 150, and the educational material profile 160) may represent five core types of solutions available to the user 105 and/or the user 107 within the semantic data structure 100. Once the solution profiles 102 are instantiated from the data entity of FIG. 1B and store specific health information, the semantic database 210 may contain hundreds, thousands, hundreds of thousands, or more instances of each of the solution profiles 102 shown in FIG. 1C. Although certain types of references are shown for each of the solution profile 102, other types of references may be included. Each reference may include a reference modification data 113, a relation status 114, and/or a designation data 115.

The professional profile 120.1 may represent a healthcare professional, including for example a doctor, physician, clinician, or other healthcare worker. The content data 104.1 may include, for example a professional designation, a personal story, a number years in practice, a preferred language, a spoken language, a set of one or more certifications, and/or a set of one or more qualifications. The set of reference attributes 110.1 includes a business reference 132.1 and a material reference 162.1. The location reference 182.1, the specialty reference 172.1, the modification data 123.1, and the tag reference 109.1 are further shown and described in conjunction with the embodiment of FIG. 1D. The professional profile 120.1 may reference, through a derivative reference 116, the services profile 150.4 as shown and described in conjunction with the embodiment of FIG. 1D. The action function 106.1 may include, for example, “make an appointment”, “call phone number”, and/or “save to my favorites”.

The business profile 130.2 may represent a business, for example a healthcare business such as a clinic, hospital, health system, and/or a medical store, a pharmacy. The content data 104.2 may include, for example a clinic description, an admission policy, a set of forms (e.g., pre-admission and/or new patient forms), and contact information for other clinic departments (such as billing inquiries). The business profile 130.2 may include a professional reference 122.2 along with a location reference 182.2 of the professional reference 122.2 to a location where the professional associated with the professional profile 120.1 works and a material reference 162.2 referencing an educational material profile 160. The business profile 130.2 may also include a technology/product reference 142.2 along with a location reference 182.2 to a location where technology, device, and/or system of the technology/product profile 140.3 is offered, and/or where a product of the technology/product profile 140.3 is offered. The business profile 120.1 may reference, through a derivative reference 116, the services profile 150.4 (via the professional profile 120.1) as shown and described in conjunction with the embodiment of FIG. 1D. The action function 106.2 may include, for example, “save as favorite”, “make an appointment”, “leave a message”, or “request visual contact”.

The technology/product profile 140.3 may be a profile representing a healthcare technology, a healthcare product, in one or more embodiments. The technology/product profile 140.3 may be a profile representing a technology, a device, a system, an apparatus, and/or a product used in providing a healthcare solution, according to one or more embodiments. Examples of technology may include medical lasers, medical imaging equipment, etc. The technology may be stationary (e.g., an CT scanner) or mobile (e.g., a portable dialysis machine as may be specified in the content data 104.3). Products can include consumable medical supplies (e.g., sold by medical suppliers), health supplements, pharmaceuticals, etc. The content data 104.3 may also include, for example a weight, a set of dimensions, a price, a warranty, a portability, and/or technical characteristics. The technology/product profile 140.3 may include a business reference 132.3 and a material reference 162.3. A hierarchical reference 175.3 may be drawn to other instances of the technology/product profile 140.3 in a more rigid and/or constrain data structure to provide hierarchical categories of products and/or technology. For example, a first technology/product profile 140.3A may be a general category (e.g., medical imaging), a second technology/product profile 140.3B may be a more specific category (e.g., MRI imaging), and a third technology/product profile 140.3C may be a specify brand and/or model (e.g., a 1.5 Tesla Magnetom Altea®). The specialty reference 170.3 and the vendor reference 192.3 are shown and described in conjunction with the embodiment of FIG. 1C. The action function 106.3 may include, for example, “contact the seller”, “schedule a virtual presentation”, and/or “request in-person show”.

The services profile 150.4 may represent a healthcare server, health services, according to one or more embodiments. The services profile 150.4 may be, in what may be similar to the technology/product profile 140.3, defined in a hierarchy of varying levels of specificity and/or categories (not shown in the present embodiment). The content data 104.4 may include, for example a service description, an estimated recovery time, an estimated patient qualification (e.g., a health requirement to undergo the service), and/or a review of a patient who received such service. The services profile 150.4 may be directly referenced by one or more other solutions profiles 102, and/or may be derivatively referenced, as shown and described in conjunction with the embodiment of FIG. 1D. The action function 106.4 may include, for example, “save for later”, and/or “request more info”.

The educational material profile 160.5 may represent an educational material, ranging from relatively informal health editorials to fully peer-reviewed publications in medical journals. The content data 104.5 may include, for example a description of a new robotic procedure, a list of one or more benefits of the procedure, a list of one or more possible complications, a technology review and description, a scientific publications referencing such procedure (which may also be referenced through a referenced through one or more material references 162). The educational material profile 160.5 may include a business reference 132.5, a technology/product reference 142.5, and/or a service reference 152.5. The educational material profile 160.5 may also include one/or more material references 162.5 to other educational material profiles 160 that may be related, a citation, supportive of information of the educational material (e.g., a validating study), and/or contradictory to information in the medical material (e.g., an opposing viewpoint, a study showing a new alternative treatment currently under FDA review, etc.). The action function 106.5 may include, for example, “switch to search”, “save to favorites”, ‘read similar materials”, and/or “contact the contributing author”.

For clarity, one of each type of solution profile 102 is shown in FIG. 1C. However, the semantic database 210 may a large number of data entities 101 as nodes of a vast network connected through references. For example, a professional profile 120 may be related to two or more business profiles 130 being that a professional associated with the professional profile 120 may work for several clinics. A business profile 130.2 may reference fifteen instances of the professional profile 120. An educational material profile 160 may be related to multiple other educational material profiles 160, for example backward citations, forward citations, and/or articles related and/or include relevant topics or content. A business profile 130 may reference hundreds of instances of technology/product profiles 140 of technology it has on-site at one or more associated locations (e.g., an MRI machine), etc.

FIG. 1D is a data entity data structure 197, according to one or more embodiments. FIG. 1D illustrates a number of other data entities 101 that may be included in the semantic data structure 100. References drawn between and/or among the solutions profiles 102 of FIG. 1C continue to be present but are shown omitted from FIG. 1D for clarity.

A specialty profile 170.6 comprises one or more service references 152.6, the service reference 152.6A through service reference 152.6N. The specialty profile 170.6 may be referenced by the specialty reference 172.1 such that each service reference 152.6 may be a derivative reference 116 of the professional profile 120.1. The reference modification data 123.1 may be used to remove services profiles 150.4 or otherwise modify data (e.g., the content 104.4) of derived information received from the service profile 150.4. The professional profile 120.1 references the tag data 108A through the tag reference 109.1A and references the tag data 108B from the tag ref 109.1B.

The business profile 130.2 may include one or more location references 182.2 to a location profile 180.7. In addition, the business profile 130.2 may modify a professional reference 122.2 with a reference modification data 113 (not shown) that designates one or more locations where the professional is present (e.g., the reference modification data 113 may take the form of or include the location reference 182.2, in one or more embodiments). Similarly, the business profile 130.2 may modify a technology/product reference 142.2 with a reference modification data 113 (not shown) that designates one or more locations where the technology is located and/or the product is available (e.g., the reference modification data 113 may take the form of or include the location reference 182.2, in one or more embodiments). The tag reference 109.2B may, as shown, may reference the same tag data 108B as the professional profile 120. This illustrates that, in general, a tag data 108 may apply to multiple types of solution profile 102.

The location profile 180.7 may include a map data 184, comprising data usable to display a location associated with the location profile 180.7 on a map, for example an address, a geospatial coordinate, etc. It should be noted that in one or more embodiments, the location profile 180 may represent a physical location (e.g., a multi-story building, a complex) that may have multiple associated businesses leasing space or otherwise operating out of the physical location.

The technology/product profile 140.3 may include a specialty reference 170.3. In one or more embodiments, only applicable services (e.g., service profiles 150.4) may be referenced directly (not shown) and/or derivatively referenced via the specialty reference 170.6. For example, the technology/product profile 140.3 representing an MRI machine may have a reference to an instance of the service profile 150.4 for medical imaging. The vender reference 192.8 may be made to the manufacturer/vendor profile 190.8. The technology/product profile 140.3 may further include one or more hierarchy references 175.3 to one or more other technology/product profiles 140 (e.g., illustrated in the present example as the technology/product profile 140.9). The manufacturer/vendor profile 190.8 may represent a technology licensor, manufacturer, distributor, dealer, and/or other supplier of one or more technologies and/or products. The manufacturer/vendor profile 190 may include on or more references to technologies and/or products, the technology/product reference 142.8A through the technology reference 142.8N. As described in conjunction with FIG. 1C, the educational material profile 160.5 may reference one or more other educational material profiles 160, illustrated in FIG. 1D as the material reference 162.5 to the educational material profile 160.10.

FIG. 1E is a data structure reference view 198, according to one or more embodiments. In the embodiment of FIG. 1E, four solution profiles 102 are illustrated with various entity references 112 draw to define various relations. Each of the references may be defined at different times, by solution profiles 102 with differing authorities, and/or by solution profiles with different statuses such as a verification status, which may result in such references being expressed (e.g., an expressed reference 118A) or unexpressed (e.g., the unexpressed reference 117).

The solution profile 102.1 references the solution profile 102.2 through the entity reference 112.1B, and the solution profile 102.2 references the solution profile 102.1 through the entity reference 112.2B. At a first time, both references may be expressed, such that traversal of the semantic data structure 100 from one solution profile 102 will find the other, and vice versa. At a later time, however, an owner and/or controller of the solution profile 102.2 may define the designation data 115 of the entity reference 112.2B. The designation data 115 of the entity reference 112.2B may, for example, explicitly designate that the solution profile 102.1 is unaffiliated with (e.g., is not related to) the solution profile 102.2, at least in the way referenced through the entity reference 112.1B. In one or more embodiments, where the solution profile 102.2 has a greater authority than the solution profile 102.1, the entity reference 112.1B would become unexpressed (e.g., the unexpressed reference 117), or in some cases may be even delated from the semantic database 210. The expression may be predetermined (e.g., at a time the user 107 associated with ownership or control of the solution profile 102.2.

In another example, the solution profile 102.2 may reference the solution profile 102.3 through the entity reference 112.2A, and the solution profile 102.3 may reference the solution profile 102.2 through the entity reference 112.3B. At a first time, neither the entity reference 112.2A or the reference entity 112.3B may be expressed because both may be unverified. This may occur, for example, when one or more solution profiles 102 of the semantic database 210 are pre-seeded with data, or their relations may be determined to be implied (e.g., through analytics, machine learning, and/or other methods) and then stored tentatively as entity references 112. At a later time, the solution profile 102.3 may be verified by the user 107 that may own or control it. In such case, the entity reference 112.3 may have associated the relation status 114 (e.g., “verified.”), resulting in expression of both the entity reference 112.2A and the entity reference 112.3B (e.g., the expressed reference 118A and the expressed reference 118B).

In another example, expression of an entity reference 112 may be a factor in one data entity 101 deriving data from another data entity 101. For example, the entity reference 112.3A may be a derivative reference 116 of the solution profile 102.2. at a first time prior to the verification, the entity reference 112.3A may be expressed with respect to the solution profile 102.3 but unexpressed as the derivative reference 116 of the solution profile 102.2 (e.g., a state which may refer to contextual expression). At a later time following the verification of the solution profile 102.3 and/or the entity reference 112.3A, the entity reference 112.3A may become the expressed reference 118C. In one or more embodiments, solution profile 102 may have a profile verification data (not shown) which applies to the entirety of the solution profile 102, or to portions thereof. For example, the profile verification data may apply to a single instance of the entity reference 112, to a group of entity references 112, and/or to other attributes such as the action function 106 and/or the content 104.

In one or more embodiments, some expression may be determined at or near the time of traversal and/or query. In one or more embodiments, a system of priority may be used to determine whether expression of an entity reference 112 should occur. For example, one solution profile 102.1 may reference another solution profile 102.2, but there may be no return reference from the solution profile 102.2 back to the solution profile 102.1 (e.g., the reference may be a unidirectional reference). In such case, a set of rules may be applied that determine priority for expression, for example based on: a profile verification data (which specified the solution profile 102 overall has been validated), an entity type data (e.g., which type of solution profile 102 makes the reference such as a business profile 130 or a technology/product profile 140, an entity size data (e.g., whether the business is a small clinic or a large health system), and/or a timestamp of the stored reference (e.g., how recently the information was stored or updated).

As just one example, a clinic may have better knowledge than a manufacturer which technological equipment they own and in which office it is physically located. A business reference 132.3 from a technology/product profile 140.3 to a business profile 130.2 as shown in FIG. 1C, which may have been initially defined manufacturer/vendor profile 190.8 (e.g., by uploading a list of businesses known to have purchased MRI machines), may be unexpressed until the business profile 130.2 is verified and/or the user 107 who owns and/or controls the business profile 130.2 has had time to review or disable the reference to the technology.

FIG. 2 illustrates the database server 200, according to one or more embodiments. The database server 200 comprises a processor 201 that is a computer processor (e.g., one or more processing cores) and a memory 202 that is a computer memory. The database server 200 is communicatively coupled to the network 199 and may further comprise a database management system 206 and a query agent 204. The database server 200 stores a semantic data structure 100 comprised of a plurality of data entities related to one another through one or more references, for example the data entities 101 and/or instantiations thereof shown and described in conjunction with FIG. 1B through FIG. 1E. With additional information such as the names, content, and relations of an actual set of real-world dataset, the semantic data structure 100 may define the semantic database 210.

The query agent 204 receives query requests (e.g., a call from the coordination server 300) and executes the query requests on the semantic database 210. For example, the coordination server 300 may transmit data requesting an entry point determination based on an entry criteria data 505. In another example, the coordination server 300 may request that a data entity 101 may be queried for extraction and return of all or a subset of the data of the data entity 101, e.g., a card data 214, an expanded card data 216, and/or a profile data 218. Such a request may be received initially from the client device 500 as the profile request data 507. In yet another example, the coordination server 300 may request that a data entity 101 is queried and its references are traversed (including traversal with varying “reference distances” from the data entity 101) to result in a traversal set 212 comprised of the set of unique identifiers 103.1 through the unique identifier 103.n. Additional data may be included in the traversal set 212A (e.g., tag data 108) to determine their relevance to the entry criteria data 505, the traversal criteria data 305, and/or any filters to be applied. In still another example, the query agent 204 may receive queries when a data card 715 is expanded and an expanded version of the card data 214 (e.g., the expanded card data 216) may be requested.

The database management system 206 is a software application that manages the semantic database 210 and/or the mapping database 220, discussed below. In one or more embodiments, the database management system may be a commercial database such as MongoDB®, Oracle®, Cassandra™, Neo4J®, etc. The semantic data structure 100 and/or the semantic database 210 may be implemented in one or more logical data models including a relational model, a columnar database, a key-value store, and/or a graph model. The database management system 206 may respond to read and write operations in the setup and maintenance of the semantic database 210.

The database server 200 may comprise a mapping database 220 that stores data mapping a first set of term values (e.g., a first terminology value 121) to a second set of term values (e.g., a second terminology value 222) form a medical taxonomy 224. In one or more embodiments, the first set of term values includes the body part value 602, the body organ value 604, the category value (e.g., the general category value 606, the specific category value 608), and the disease value 610.

In one or more embodiments, the mapping database 220 may store one or more tag data 108 that may engrain the mapping from one or more terms to one or more other terms, and which may be referenced by one or more of the data entities 101 of the semantic database 210 (alternatively or in addition, the tag data 108 may be stores as instances of the data entity 101 within the semantic database 210 including within the data entities 101). In one or more embodiments, the tag data 108 comprises a unique identifier 111, one or more terminology values 121 (shown as the “term value 121”) and one or more terminology references 119 (shown as the “term ref 119”). The terminology reference 119 references a terminology value 222 within a medical taxonomy 224 to create a terminology mapping. For example, the terminology value 121 may be “ophthalmologist” and the terminology value 222 may be “eye doctor”. In another example, the terminology value 121 may be “optical coherence tomography” and the terminology value 222 may be “eye exam”. In one or more embodiments, each tag data 108 may be “appended” to data entities 101 (e.g., through the tag reference 109) manually and/or automatically, for example when information is initially added to the data entity 101 and/or the data entity 101 is claimed. In a specific example, the tag data 108 with a terminology value 121 of “spinal cord” may be determined to be applicable to a data entity have a content data 104 with the word “laminectomy” (which may be the terminology value 222). The tag mapping module 208 comprising computer readable instructions that when executed (e.g., on the processor 201) map the tag data 108 comprising at least one of the body part value 602, the body organ value 604, the category value (e.g., the general category value 606, the specific category value 608), and the disease value 610, to a terminology value 222 of a medical taxonomy 224 through a tag reference 109 to the tag data 108.

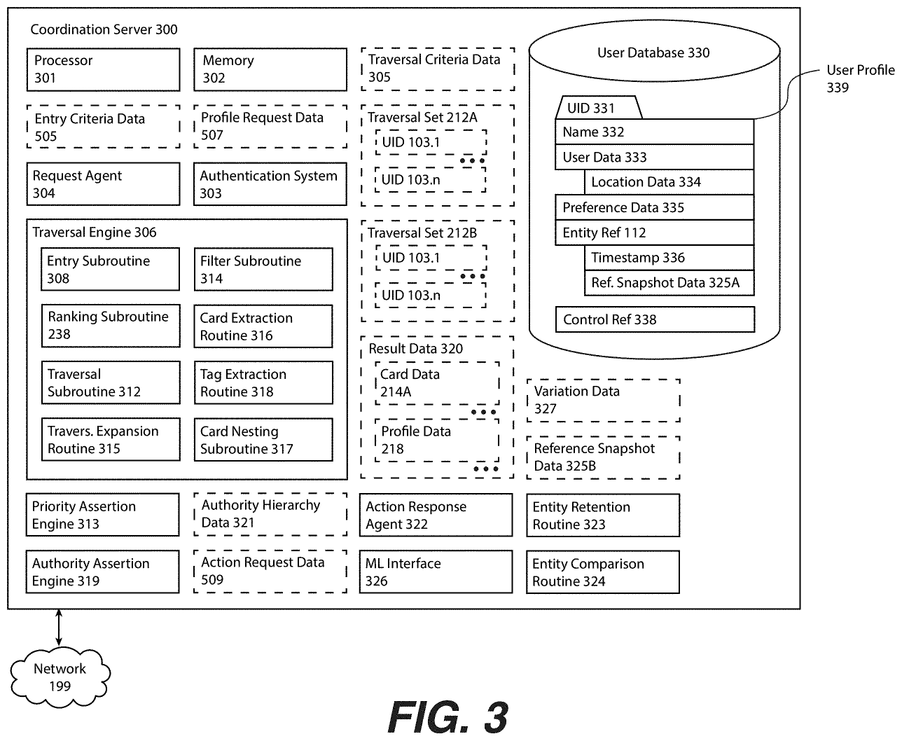

FIG. 3 illustrates the coordination server 300, according to one or more embodiments. The coordination server 300 comprises a processor 301 and a memory 302. A request agent 304 receives requests from a client device 500, for example for initial starting point (e.g., entry into the semantic data structure 100), continued exploration (e.g., traversal of the semantic data structure), retrieval of specific information of a data entity 101 (e.g., a card data 214 and/or a profile data 218), and/or other requests. In one or more embodiments, the request agent 304 further comprises computer readable instructions that when executed receive and/or process an entry criteria data 505 (e.g., received over the network 199 from the client device 500). In one or more embodiments, the request agent 304 further comprises computer readable instructions that when executed receive and/or process a profile request data 507 (e.g., received over the network 199 from the client device 500).

An authentication system 303 comprises computer readable instructions that when executed authenticate at least one of a user 105 and/or a user 107 and a client device 500 of the user 105 and/or the user 107. The authentication may be single factor or multi-factor as may be known in the art. The authentication may be performed to gain access to, control and read and/or write to a user profile 339 (e.g., by the user 105A of FIG. 1A). The authentication may also be performed to control a data entity 101 including to help define portions of the semantic database 210 (e.g., the user 107B of FIG. 1A).

The coordination server 300 includes a traversal engine 306 comprising computer instructions utilized to enter, traverse, and return results from the semantic data structure 100. In one or more embodiments, the traversal engine 306 may help enable a smooth, seamless navigation and/or exploration through the semantic database 210 from the perspective of the user 105A interacting with the user interface of the client device 500A. The traversal engine 306 may comprise computer readable instructions that when executed (i) determine an entry point in the semantic data structure 100 comprising one or more data entities 101 as entry nodes, (ii) determine one or more entry nodes related to each data entity 101 and traverses references to determine related nodes; (iii) determine relevancy of the related nodes based on an entry criteria data 505 and/or a traversal criteria data 305; (iv) filter results based on preferences (e.g., the preference data 335) or other factors; (v) returns results to the client device 500 (e.g., the result data 230); and/or (vi) receive additional requests for data of specific data entities 101, resulting returning data of those data entities 101 followed again by further traversal, relevancy determination, filtering, and/or results.

In one or more embodiments, the traversal engine 306 may comprise a number of subcomponents each with distinct and/or overlapping functionality. An entry subroutine 308 comprises computer instructions that determines one or more nodes of the semantic data structure 100 for initial entry. In one or more embodiments, the entry subroutine 308 may comprise computer readable instructions that when executed initiates a query to the semantic data structure 100 to receive a first set of unique identifiers 103 (e.g., the traversal set 212) of a plurality of data entities 101 each comprising a tag data 108 matching terms and/or one or more tag data 108 of the entry criteria data 505. An index of the semantic database 210 may be utilized to initially find matching tag data 108. For example, in one or more embodiments, an initial entry node is determined by the closest match of tag data 108 specified in the entry criteria data 505 to the most tag data 108 of solution profiles 102. For purposes of an ongoing example, the entry subroutine 308 may have generated a first traversal set 212A comprising the UID 103.1 through the UID 103.n.

A traversal subroutine 312 may comprise computer readable instructions that when executed that coordinate and manage traversal of references within the semantic database 210 and the building of traversal sets 212. In one or more embodiments, the traversal subroutine 312 may determine how far references should be followed (e.g., how many reference “jumps” from a given data entity 101), how many data entities 101 should be queried for extraction of data, and/or other steps and functions related to traversing the semantic data structure 100. For purposes of an ongoing example, the entry subroutine 308 may have further contributed to the first traversal set 212A comprising the UID 103.1 through the UID 103.n if not enough entry nodes were initially determined for return to the user 105A. The traversal subroutine 312 may assess relevance in real time during traversal, with data sufficient for the relevance determination returned from the database server 200 along with the unique identifier 103 of the associated data entity 101. As just one example, relevance may be determined to be any data entity 101 connected through a reference to an entry node, or any data entity 101 up to three references away (e.g., a “reference distance”) that shares at least one tag data 108 of the entry criteria data 505.

The traversal subroutine 312 may also traverse references in response to a query of a specific data entity 101 and/or solution profile 102, as may be specified in the profile request data 507. In one or more embodiments, the traversal subroutine 312 comprises computer readable instructions that when executed traverse within the semantic data structure 100 one or more references (e.g., entity references 112) of a solution profile 102 to a set of solution profiles 102, and store a set of unique identifiers 103 (e.g., a traversal set 212C, not shown) comprising the set of solution profiles 102 referenced through the one or more references upon receiving the first profile request (e.g., via the profile request data 507).

The filter subroutine 314 comprises computer readable instructions that when executed apply one or more filters that may further limit results that may have a lower probability of relevance. For example, the filter may relate to a certain preference specified by the user 105 (“I prefer a female professional”), or excluding results associated with locations outside a certain radius centered on an address of the user 105. In one or more embodiments, the filter subroutine 314 remove a first subset of the first set of unique identifiers 103 based on a location data 334 associated with the user 105A stored in the user profile 339 and the preference data (e.g., the preference data 335) of the user 105A to result in a second set of unique identifiers (e.g., the traversal set 212B). It should be noted that while the traversal set 212A and the traversal set 212B are shown including the unique identifier 103.1 through a unique identifier 103.n, the traversal set 212B generally will include less than all of the unique identifiers 103 specified in the traversal set 212.

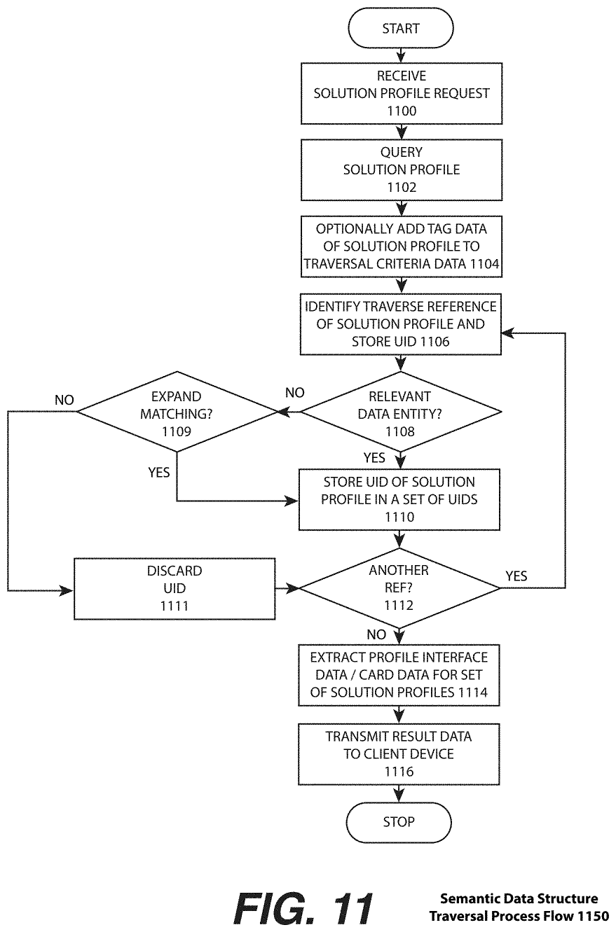

The traversal expansion routine 315 expands the extent of traversal and/or reduces the amount of filtering to introduce random and/or different results. This introduction of variation may allow the user to transition to a new part of the semantic data structure 100 to which they may otherwise have not been exposed based on their initial entry criteria and/or traversal data. The traversal expansion routine 315 may also be utilized to ensure there are enough results within the result data 320, for example to ensure enough instances of the card data 214 are transmitted to populate one data card 715 on each row of the entry point interface 700 of FIG. 7 (e.g., four technology/product profiles 140). In one or more embodiments, the traversal subroutine 312, the filter subroutine 314, and the traversal expansion routine 315 may run concurrently, building the traversal set 212A at the same time each unique identifier 103 is filtered to result in the traversal set 212B, and, in the event the traversal set 212B is below a threshold value, the traversal expansion routine 315 may expand criteria used in traversal of the traversal subroutine 312 to continue to generate additional results. Additional aspects that may be utilized are further described in conjunction with the embodiment of FIG. 11. Expansion may also be accomplished, alternatively or in addition, by determining a new tag data 108 in which the user 105 may be interested and adding the new tag data 108 to the traversal criteria data 305, as shown and described below.

The card extraction routine 316 comprises computer readable instructions that when executed initiate query and extract data of a data entity 101 by calling the semantic database 210 with a unique identifier 103 of the data entity 101 and may then transmit the results back to the client device 500 within the result data 320. The result data 320 comprises one or more card data 214 and/or one or more instances of the profile data 218. The card extraction routine 316 may include in the query a designation of how much data to return, for example a card data 214 that may be a cursory dataset an expanded card data 216, and/or a complete instance of the profile data 218. In one or more embodiments, the card extraction routine 316 may comprise computer readable instructions that when executed transmit to the client device 500 of the user 105 a card data 214 for each solution profile 102 associated with the a set of unique identifiers 103 (e.g., the unique identifier 103.1 through the unique identifier 103.n of the traversal set 212B), for example as the result data 320. In the present example, the user 105 may then have been presented semantically related data entities 101 traversable by the user 105 within the semantic data structure 100 to increase the probability of determining a data entity 101 storing healthcare data applicable to the user 105.

The tag extraction routine 318 comprises computer readable instructions that when executed extracts one or more tag data 108 stored in and/or referenced by a data entity 101. For example, where the user 105 demonstrates interest in several solution profiles 102 which all have the same or similar tag data 108 associated, that tag data 108 may be utilized to increase relevance during traversal of the semantic data structure 100 (e.g., as may be applied by the traversal subroutine 312). In one or more embodiments, a traversal criteria data 305 may include tag data 108 and/or other data helping to narrow in on applicable instances of the solution profile 102 within the semantic data structure 100. In one or more embodiments, the tag extraction routine 318 comprises computer readable instructions that when executed: (i) initiate a traversal criteria data 305 (which may optionally initially comprise the entry criteria data 505); (ii) extract a tag data 108 a solution profile 102 upon receiving a profile request data 507 to retrieve data of the solution profile 102 (or another interaction such as triggering an action function 106); and (iii) store the tag data 108 of the first solution profile in the traversal criteria data 305. A subsequently determined set of unique identifiers 103 (e.g., the traversal set 212C) may comprise one or more solutions profiles 102 referencing the tag data 108.

A card nesting subroutine 317 may combine the information of one or more data entities 101 such that they may be presented as a combined concept and/or data card 715, while remaining independent within the semantic data structure 100. This may be helpful to create pseudo-entities being that some users 105 may not typically understand the boundaries of the underlying data entities 101 from which the data cards 715 derive. The nesting can also be utilized to more explicitly show relations of affiliation and/or trust while keeping modeling and/or control separate. In just one example, this “nesting” of information may be useful when presenting the service profile 150, where a user 105A may be unlikely to interact with a “service” card having no apparent offeror. For example, the data card 715 of a service profile 150 may be populated with the card data 214 of the service profile 150, but also includes some card data of a professional profile 120 having a reference (and/or a derivative reference 116 via the specialty profile 170) to the service profile 150. In one or more embodiments, the card nesting subroutine 317 comprising computer readable instructions that when executed extract a card data 214A of the first solution profile 102.1, extract the card data 214B of a second solution profile 102.2, and generate a nested data card (e.g., the nested data card 720 of FIG. 7) that incorporates at least a portion of the card data 214A of the first solution profile 102.1 into the card data 214B of the second solution profile 102.2.

A ranking subroutine 238 may prioritize results for the user interface, for example ordering and determining which data cards 715 should be displayed in limited space, higher on a scrolling list, receive visual emphasis, etc. In one or more embodiments, the ranking subroutine 238 comprising computer readable instructions that when executed determine a presentation order of the card data 214 (e.g., as data cards 715) for each data entity 101 associated with a set of unique identifiers 103 (e.g., returned in a result data 230) on a display application 508 running on the client device 500. The display order and/or priority may be communicated as part of the result data 230, or may even be calculated on the client device 500.

In one or more embodiments, expression or unexpression of certain references within the semantic data structure 100 may be determined prior to, during, and/or after generation of the traversal set 212. Such expression may take place, in one or more embodiments, because certain data entities 101 may have more authority than others to define references, and/or various factors may affect the strength and/or quality of the reference.

As used herein, a “stored reference” is a reference which is stored within the reference attributes 110 of a data entity 101, and can be either expressed or unexpressed with respect to a given user 105 generating a query. An unexpressed reference is one in which the reference is treated as non-existent for purposes of the query (and/or all queries from a user 105) and will therefore generally not be traversed or utilized to extract derivative data. An expressed reference is one in which the reference is treated as in existence for purposes of the query (and/or all queries) and may possibly be traversed (e.g., by the traversal subroutine 312) and/or utilized to import derivative data.

In one or more embodiments, a priority assertion system 313 determines priority over conflicting and/or unidirectional references within the semantic data structure 100, especially upon query. For example, a first data entity 101.1 may have a first stored reference (e.g., an entity reference 112.1A) to a second data entity 101.2 and a second stored reference (e.g., an entity reference 112.1B) to a third data entity 101.3, where both relations are known to be mutually exclusive (e.g., a clinic belonging to two distinct HMOs). In such case, a priority determination may occur to resolve the apparent conflict, for example where the data entity 101.3 also references the first data entity 101.1, indicating that the information is more likely reliable and/or accurate. In such case the entity reference 112.1A may be unexpressed and the entity reference 112.1B expressed.

In one or more embodiments, the priority assertion system 313 may comprise computer readable instructions that when executed determine that a stored reference within in the semantic data structure 100 as an attribute of a first solution profile 102 is a unidirectional reference to a second solution profile 102. The priority assertion system 313 may further comprise computer readable instructions that when executed determine the stored reference is prioritized based on: (i) a profile verification data, (ii) an entity type data (e.g., a user 107 controlling a business profile 130 may know whether they have a piece of technology better than a user 107 controlling manufacturer/vendor profile 190), an entity size data (e.g., a business with fewer affiliated professionals may have better knowledge about certain relationships than a business with thousands of affiliated professionals), and a timestamp of the stored reference (e.g., how recent the information is and whether it was defined before or after a new instance of the user 107 took control of the data entity 101). The priority assertion system 313 may further comprise computer readable instructions that when executed determine the stored reference of the first solution profile 102 is an expressed reference, such that a unique identifier of the first solution profile 102 is included in a set of unique identifiers 103 for transmission of a card data 214 to the client device 500 of the user 105. Variable expression of stored references based on prioritization of information may increase probability of modeling accuracy by responding to dynamically changing information in the semantic database 210.

An authority assertion system 319 may similarly determine which solution profiles 102 (and/or their associated controlling instance of the user 107) have authority to define references in the semantic data structure 100 and therefore control the formation of asserted relations. In one or more embodiments, the authority assertion system 319 comprises computer readable instructions that when executed: (i) receive a request to delete a first reference (e.g., an entity reference 112.1) stored within the semantic data structure 100, the first reference drawn from the first solution profile (e.g., a solution profile 102.1) to the second solution profile (e.g., a solution profile 102.2) to assert a relation between the first solution profile and the second solution profile; (ii) compare a unique identifier 103 of the first solution profile and a unique identifier of the second solution profile; (iii) reference an authority hierarchy data 321; (iv) determine the second solution profile is higher in the authority hierarchy data; and/or (v) delete the first reference in the first solution profile and/or designate the first reference as an unexpressed reference 117 within the semantic data structure 100 (e.g., through the designation data 115). The authority hierarchy data 321 may specify, for example, that a professional profile 120 has the power to define an entity reference 112 with designation data 115 explicitly negating a relationship to a business profile 130 and/or deleting the professional reference 122 of the business profile 130 to the professional profile 120 (even if the business profile 130 is not owned or controlled by the user 107 owning and/or controlling the professional profile 120). In another example, a verified professional profile 120 with a certain activity over a previous 12-month period may be able to directly define a material reference 162 from a first educational material profile 162.1 to a second educational material profile 162.2.

Upon the user 105A reviewing the result data 320, the user 105 may either generate an additional profile request data 507 (which may lead to another instance of the result data 320 generated), and/or may trigger an action function 106 of one or more of the data entities 101. When triggering an action function 106, the user 105 may select an “action button” on a data card 715 which generates an action request data 509 which may include the unique identifier 103 of the associated data entity 101. The action request data 509 may be received over the network 199 by the action response agent 322, which may then initiate query of the associated data entity 101 for execution of the action function 106. In one or more embodiments, the action response agent 322 receives the action request data 509 and initiates an action associated with a solution profile 102. For example, the action may be “change the area of search”.

The coordination server 300 may further include an entity retention module 323 and/or an entity comparison routine 324, according to one or more embodiments. The entity retention module 323 may be utilized to save one or more data entities 101 for later query by a user 105. From the perspective of the user 105, in one or more embodiments, it may appear as if the user 105 saved the “card” (e.g., the data card 715 of FIG. 7), which may be accessible through part of a user 105's profile when the user 105 is authenticated. In one or more embodiments, the entity retention module 323 may comprise computer readable instructions that when executed store a reference from the user profile 339 to a unique identifier 103 of a first solution profile 102 to enable the user 105 to re-query the data card 715 of the first solution profile 102 (e.g., the data card 715 of the card data 214 of the first solution profile 102). The entity retention module 323 may further comprise computer readable instructions that when executed generate at a first time a first reference snapshot data 325 of one or more references (e.g., the entity reference 112) referencing a solution profile 102 saved by the user 105, the first reference snapshot data 325 (e.g., a reference snapshot data 325A) comprising a unique identifier 103 one or more data entities 101 referencing the solution profile 102.

An entity comparison routine 324 may be utilized to compare a state of a data entity 101 at a first time and a state of a data entity 101 at a second time (e.g., one hour later, one week later, one month later). From the perspective of the user 105, the data cards 715 may be compared. In one or more embodiments, the entity comparison routine 324 comprises computer readable instructions that when executed generate at a second time a reference snapshot data 325 (e.g., the reference snapshot data 325B) of one or more references drawn to a solution profile 102 saved by the user 105 by re-traversing, upon receiving the second profile request (e.g., the profile request data 507), one or more references of the solution profile 102. The second reference snapshot data 325 may comprise a unique identifier 103 of one or more data entities 101 referencing the solution profile 102 (e.g., “incoming references”). The entity comparison routine 324 may further comprise computer readable instructions that when executed determine a variation of the first profile data 218 at the first time and the second time and transmit a variation data 327 to the client device 500A of the user 105A. The variation data 327 may be, for example, a simple report in narrative report (“the professional is now affiliated with a new clinic”), markup on the data card 715 (including strikethrough or underline, as if redlined), and/or highlighted text showing changes and/or new additions. Although not shown, an update of any changes to a saved data entity 101 may also be sent at the time they occur to the client device 500 of the user 105, as further described in conjunction with the embodiment of FIG. 13.

The embodiment of FIG. 3 further illustrates a user profile database 330 (although the user profile database 330 may also be stored on a distinct server). The user profile database 330 comprises a plurality of user profiles 339. Each user profile 339 may represent any natural person user, including users 105 generally searching the semantic database 210 and those user 107 defining data entities 101 of the semantic database 210. For example, the user 107B of FIG. 1 may be a professional who then is able to setup and/or claim a preexisting professional profile 120 that represents themselves as a “solution” within the semantic database 210.

The user profile 339 includes a unique identifier 331, a name 332, a set of user data 333 (which may include a location data 334 such as an address and/or a geospatial coordinate), and/or a set of preference data 335 which may be selected by the user 105 or possibly inferred from activity of the user 105 or other data sets. For example, the preference data 335 may include “concierge service”. The user profile 339 may also store a reference to the data entity 101 the user is “saving” (e.g., the entity reference 112), a timestamp 336, and the reference snapshot data 325 at the time of the timestamp 336 (shown here as the reference snapshot data 325A). A control reference 338 may store data specifying one or more data entities 101 a user 107 generally owns and/or controls.

The data generated by the coordination server 300 may also be useful for big data analysis and/or machine learning. In one or more embodiments, a machine learning interface 326 may transmit data to and/or receive data from a machine learning system, for example the machine learning server 400.

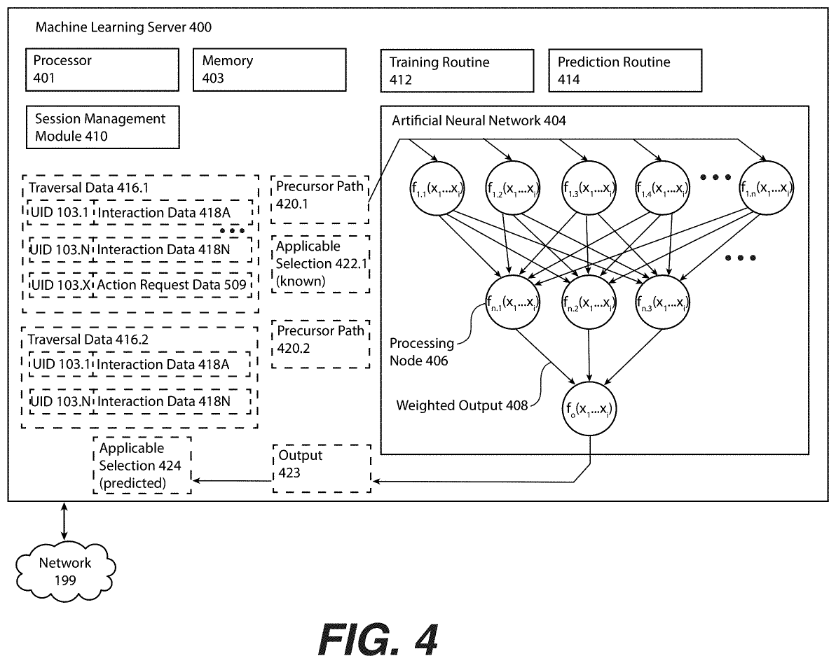

FIG. 4 illustrates a machine learning server 400, according to one or more embodiments. The machine learning server 400 includes a processor 401 and a memory 402. An artificial neural network 404 that may be used to apply a machine learning process (e.g., as may be colloquially referred to as “artificial intelligence”) to recognize patterns,

The artificial neural network 404 comprises one or more processing nodes 406 (distinct from nodes of the semantic data structure 100). Each processing node 406 may have a series of inputs and/or a series of outputs, where each processing node 406 may include a mathematical function specifying a weight of an input (e.g., the weighted output 408). A model set of processing nodes 406 are shown in FIG. 4 for illustration, but the total number of processing nodes 406 may include hundreds, thousands, or more.

In one or more embodiments, including the embodiment of FIG. 4, each processing node 406 stores a function that accepts a set of one or more inputs, either as initial inputs or from one or more additional processing nodes 406. For example, the function ƒ_{1.1} refers to the first node of the first row, and may accept at least one input x₁, apply a constant ci or other value to the input (not shown), and generate an output value, then propagated the output value to one or more processing nodes 406 further down (e.g., “deeper”) within the artificial neural network 404, including to any “hidden layers” as may be known in the art. The processing node 406 bearing the function ƒ_{n.1} refers to the first node of the nth row, and accepts inputs from the row immediately above (as shown, ƒ_{1.1} through ƒ_{1.n}). The output node ƒₒ may receive inputs and output one or more values corresponding to a recognized pattern, e.g., a prediction. This may illustrate just one way in which the artificial neural network 404 can be defined, and other methods known in the art will be apparent to one skilled in the art.

A session management module 410 comprises computer readable instructions that when executed initiates a training session and/or initiates a prediction session, and receives a traversal data 416 through the network 199 (e.g., the traversal data 416.1). The traversal data 416 includes data about one or more interactions the user may have with the semantic database 210 and/or the user interface displaying data extracted from the semantic database 210. For example, a unique identifier 103.1 of a data entity 101 is associated with an interaction function 418A specifying an interaction that the user 105 and/or the user profile 339 of the user 105 effected. Interactions may include, for example, a data card 715 expansion, generation of a profile request data 507, a tag data 108 added to the traversal criteria data 305, and other data. The traversal data 416.1 may also include an action request data 509 associated with the unique identifier 103.x.

A training routine 412 comprises computer readable instructions that when executed may receive the traversal data 416 and apply the traversal data 416 to train the artificial neural network 404. The traversal data 416 may be utilized to generate the precursor path 420, which may be one or more interactions of the user 105 prior to generating the action request data 509. The traversal data 415 may also include the action request data 509 which may be utilized as the applicable selection 422. In one or more embodiments, the precursor path 420.1 may be a set of unique identifiers 103 and/or tag data 108 associated with data entities 101 with which the user 105 may have strongly interacted before being led to the data entity 101 in which the action function 106 was triggered. The precursor path 420.1 may be input into the artificial neural network 404, along with the applicable selection 422 as a “correct” output, to train the artificial neural network 404. In one or more embodiments, the training may update the weighted outputs 408 of one or more processing nodes 406. The artificial neural network 404 may be trained on tens or millions of instances of the traversal data 416. Interactions may be normalized prior to utilization as a precursor path 420 to account for differences in user interaction level.

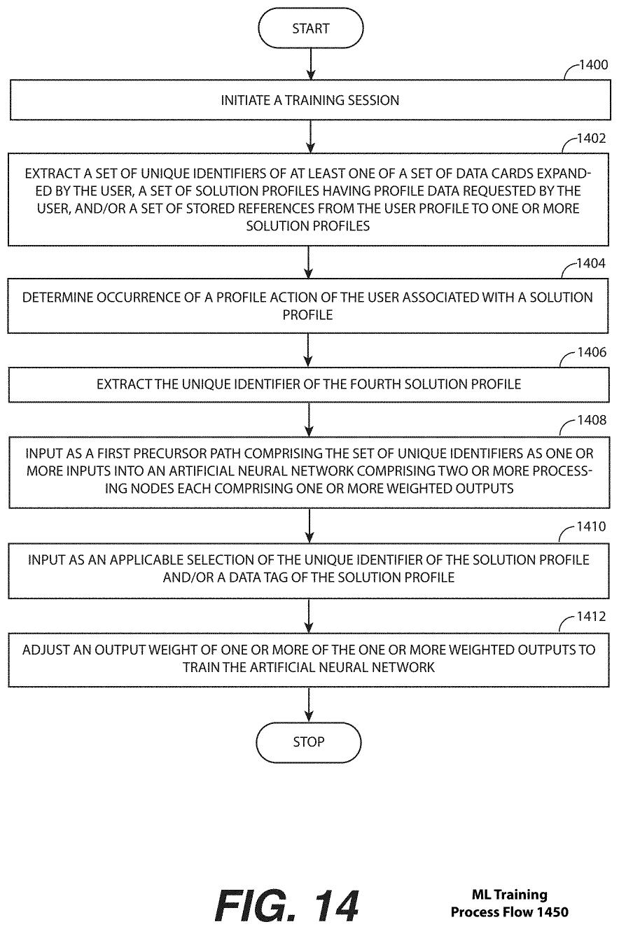

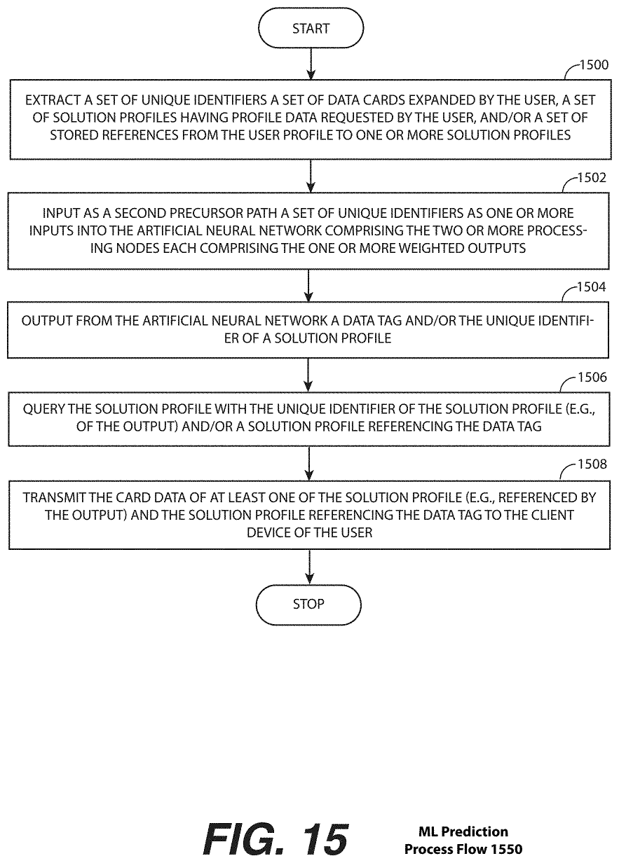

The machine learning server 400 comprises a prediction routine 414. The prediction routine 414 comprises computer readable instructions that input a traversal data 416 (e.g., the traversal data 416.2) into input processing nodes (e.g., the uppermost processing nodes, which are unlabeled) of the artificial neural network 404 as the precursor path 420.2. The output 423 may be generated, which may include data useful in finding an applicable solution profile 102 for the user 105. An applicable selection 424 may include data usable to determine a unique identifier 103 of the data entity 101. The unique identifier 103 of a data entity 101 may then be transmitted through the network 199 to the coordination server 300 (where it may result in query of the data entity 101, extraction of the card data 214, and presentation of an associated data card 715). An applicable selection 424 may also include a tag data 108 that may be applicable to the search and/or exploration of the user 105 (and which may be optionally communicated back to the coordination server 300 through the network 199, for example to be incorporated into the traversal criteria data 305. An example machine learning process is further shown and described in conjunction with the embodiment of FIG. 14 and FIG. 15.

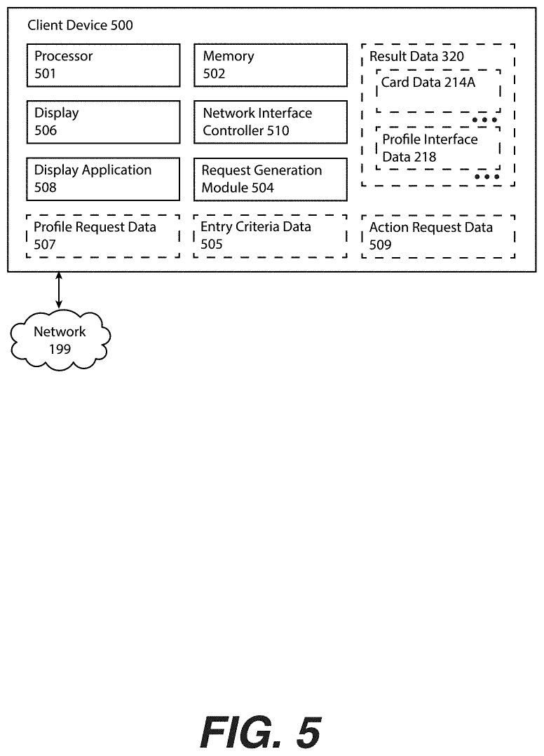

FIG. 5 illustrates a client device 500, according to one or more embodiments. The client device 500 may be, for example, a desktop computer, smartphone, mobile device, tablet device, laptop computer, or other suitable computing device. The client device 500 includes a processor 501 and a memory 502. The client device includes a display 506 (e.g., an LCD screen) that may be able to display a graphical user interface. A display application 508 is an application receiving data from (e.g., from the coordination server 500) and organizing and/or displaying the result data 320 in various ways, for example as shown and described in conjunction with FIG. 7. In one or more embodiments, the display application 508 may be a web browser, a desktop application, and/or a mobile application (e.g., an “app”). A network interface controller 510 enables communication through the network 199. A request generation module 504 comprises computer readable instructions that when executed generate the profile request data 507, generate an action request data 509, and/or generate and/or select one or more components of and the entry criteria data 505 (as further shown and described in conjunction with the embodiment of FIG. 6)

FIG. 6 is a data structure entry view 600 illustrating generation of the entry criteria data 505 usable for determination of an entry point into the semantic data structure 100, according to one or more embodiments. In one or more embodiments, a workflow for selection of one or more pieces of data usable in generating the entry criteria data 505 may be presented to the user 105 on a display of the client device 500. In one or more embodiments of FIG. 6, a workflow may initially present a choice between selecting a body part and/or a body organ from a menu and/or a graphical diagram. For example, a graphical model of the human body may be presented with selectable (e.g., clickable) elements. The user 105 may be able to select one or more body parts and/or body organs which they are familiar with, or, for example, a specific location where they are experiencing a symptom and/or pain. In one or more embodiments, the workflow may first present a human body from an exterior view to receive a selection of a body part (e.g., selecting a body part value 602), then present the human body as at least a partial transparent view permitting selection of an internal and/or external body organ (e.g., selecting a body organ value 604). A set of computer readable instructions running on the client device 500 may then store a body part value 602 and/or a body organ value 604.

The user 105 may also select one of a number of categories. The user 105 may select a general category value 606 which may generally apply to healthcare. For example, the general category value 606 may be data that specifies “diagnosis”, “treatment”, “surgery”, “urgent”, “pain”, “trauma”, “cosmetic”, “imaging”, “pediatric”, “geriatric”, “family care”, “nutrition”, and/or “natural medicine”. The user 105 may next select a specific category value 608, where the options for the specific category value 608 may be refined and/or filtered based upon the body part value 602 and/or the body organ value 604. For example, where the user selects the immune system as a body organ, the specific category value 608 presented may include “treatment of the immune system”, “diagnosis of the immune disease”, and/or “laboratory facilities with immunology tests”. As used herein, the general category value 606 and the specific category value 608 may be collectively referred to as a “category value.”

In one or more embodiments, the user 105 may also have the option of selecting a disease value 610. Similar to the specific category value 608, the disease value 610 options may be filtered based on the body part value 602 and/or the body organ value 604 (and possible other selections, including the specific category value 608).

It should be noted that some of the body parts, organs, and/or categories may overlap, or may be described at varying levels of specificity. In one or more embodiments, this may create a flexible, non-ridged system of entry into the semantic data structure 100 due to each user 105 being able to utilize language that he or she knows best which may then be mapped to a more rigid medical taxonomy through use of the tag data 108.

A property data 612 of the user 105 may also be selected either automatically and/or manually. For example, an age group (e.g., age 60-65) which may be selected by the user 105 as part of the workflow, or may be automatically extracted from the user profile 339. The property data 612, for example, can be a gender, an age group, a disease value 610 already associated with the user profile 339, a location data 334, service card data, and/or other data. As shown, the user 105 may also simultaneously submit a preference data 335.

The entry criteria data 505 may comprises data specifying the manual selections of the workflow and/or any automatic selections. For example, the entry criteria data 505 may be assembled in JSON format and transmitted over the network 199 to the coordination server 300 from the client device 500. Alternatively or in addition, the body part value 602, the body organ value 604, the disease value 610, the general category value 606, the specific category value 608, and/or and the property data 612 may be sent and the entry criteria data 505 assembled as a server-side function (e.g., on the coordination server 300), possibly after automatic inclusion of additional data (e.g., the property data 612 extracted from the user profile 339 and/or determination of preference data 335).

FIG. 7 illustrates a data structure traversal interface view 750, according to one or more embodiments. According to one or more embodiments, each of the views of FIG. 7 may be an example of a human-readable form of the result data 320 at various stages of entry and traversal of the semantic data structure 100, as may be rendered by this display application 508 as a graphical user interface on a display 506 the client device 500. In the present embodiment, it will be assumed for purposes of providing an example that the user 105 selected an entry criteria data 505 focused on the immune system, and specifically allergic reactions. For example, the user 105 may assume they are breaking own in hives and occasionally having trouble breathing due exposure to mold at their new workplace.

According to one or more embodiments, an entry point interface 700 illustrates a result data 230 after initial entry into the semantic data structure 100. The result data 230 may generated (e.g., through execution of computer readable instructions of the coordination of the entry subroutine 308, the traversal subroutine 312, and/or the traversal expansion routine 315) such that the result data 320 includes at least four solution profiles 102 from the five categories shown in the embodiment of FIG. 1C (e.g., a professional profile 120, a business profile 130, a technology/product profile 140, a service profile 150, and an educational material profile 160). In the entry point interface 700, only three rows of data cards 715 are shown, with rows accessible through scrolling or other user interface interactions. Each of the data cards 715 may be presented in their “condensed” form. The result data 230 may include a card data 214 extracted for each of the solution profiles 102. For example, from a service profile 150.1 may be extracted the card data 214.1 which may be rendered as the data card 715.1.

Each of the data cards 715, including in their condensed form, may include what may be considered to be the most succinct summation and/or essential information to assist the user 105 in determining whether the associated solution profile 102 needs to be more closely reviewed. Essential data may be designated in the data entity 101 or determined by one or more processes upon query of the data entity 101. A similar designation may be included for a second tier of important, but possibly less essential for rapid assessment or reckoning by the user 105, information reserved for an expanded form.

In one or more embodiments, a data card 715 associated with a professional profile 120 may include as its essential data: professional designation, specialty, review score. In one or more embodiments, a data card 715 associated with a business profile 130 may include as its essential data: type of business, contact info, review score, hours of operations.

The card interaction view 702 illustrates a continuation of the entry point interface 700 in which the user has scrolled down (exposing two additional rows that include data cards 715 of business profiles 130 and data cards 715 of educational material profiles 160. The user 105 may then decide to select the data card 715.15 (e.g., displaying the card data 214.15 associated with a business profile 130.15), for example by clicking on an “expand” option displayed on the data card 715.15 (not shown). The data card 715.16 may then “unfold” into the expanded data card 716.15 with additional important information. In one or more embodiments, the expansion may be an interaction which may be used to generate an interaction function 418 usable in machine learning, as shown and described in conjunction with the embodiment of FIG. 4. The data card 715 and/or the expanded data card 716 may also include images, movies, virtual reality (e.g., a short virtual tour of a doctors' office), augmented reality (e.g., a model of a piece of medical technology projected on the desk of the user 105), and/or other multimedia. In one or more embodiments, a query against the semantic database 210 for the expanded card data 216 may occur just in time upon a request to expand the data card 715 (e.g., the selection may generate a profile request data 507) which may save bandwidth and/or computing resources, especially for high bandwidth multimedia.

In one or more embodiments the user 105 may freely expand and condense data cards 715 until they find what may be perceived by the user to be the most interesting and/or appropriate starting point for a search. In one or more embodiments, this process may permit the user 105 to find their own starting point in solving a health problem or health challenge without a barrier of medical jargon or rigid predetermined taxonomies created from the perspective of a single stakeholder (e.g., a pharmaceutical company, a specific health system's service listing based on specialty and then clinician, etc.). Use of the entry point interface 700 and the expanded data card 716, from the perspective of the user 105, may result in a free flowing gathering of information without having to navigate a taxonomy and repeatedly return to a starting point (e.g., click “back” on a browser application) when incorrect or inapplicable information results.

In the present example, the user 105 may assess several data cards 715 before selecting the data card 715.7 (associated with a professional profile 120.7), for example a “view profile” option. Following the selection, the client device 500 may generate a profile request data 507 a greater amount of information from the professional profile 120.7, and the user interface may re-organize to present and emphasize the information of the professional profile 120.7 as the main focus of the user 105.

The profile traversal interface 704A illustrates presentation a profile interface 718.7 of profile data 218 from the professional profile 120.7, as returned in an instance of the result data 320. The profile interface 718.7 may be more detailed and/or include more derived information, visuals, multimedia, and action buttons (e.g., connected to action functions 106) relative to the data card 715 and/or the expanded data card 716, and/or include one or more UI elements for navigation to organize such information (e.g., a tab for “credentials”, a tab for “locations”).

In addition, as shown and described throughout the present embodiments, semantically related data entities 101 may be determined through traversal of semantic data structure 100. In the present example, such related data entities 101 result in: (i) the data card 715.3 (originally presented in the entry point interface 700), (ii) the data card 715.21 (a data card 715 for a business not yet presented to the user 105 as a data card 715, e.g., a different clinic where the professional associated with the professional profile 120.7 also works), and (iii) a data card 715.22 (a new educational material). A map 705 may include a mapped location associated with each data card 715 viewable on the profile traversal interface 704A, although some data cards 715 may not have an associated location (e.g., the data card 715.22 that displays information of an educational material) or may have multiple locations (e.g., each location associated with the professional profile 102.17, for example two clinics, a doctor's office, and a hospital.

Also illustrated in the profile traversal interface 704A, the data card 715.3 may be modified through nesting, such that the name of the professional associated with the professional profile 120.7 is incorporated into the service profile 150.3 (e.g., the data card 715.7 is “nested” within the data card 715.3). Such nesting, which may be determined randomly or according to a set of rules, may help present information in a way that is compelling, comfortable, or otherwise able to increase assessment and/or interaction with a data card 715. For example, the user 105 may feel uncomfortable selecting a “service” which no professional “attached,” because it may subconsciously feel more like an advertisement. On the other hand, after seeing the profile of a professional (e.g., viewing the profile interface 718.7), the user may be more likely to select a data card 715 representing a service as long as the professional's name appears in association with the service. Similar combinations can occur with any two (or more) solution profiles 102.

In the present example, the user 105 may expand the data card 715.22 that may represent an educational material profile 160. The expanded data card 716.22 may include a subtitle, an abstract, a summary, an introduction section, and/or the first paragraph of an editorial article. Returning to the present specific example, the article may explain that allergic reactions to food can develop later in life, and sometimes suddenly. Even without deciding to read the entire educational material, the user 105 may have learned an important piece of information, for example causing the user 105 to realize that the user 105 recently purchased a large amount of a certain food (e.g., peanut butter) and has been eating it every day for the past two weeks.

The user 105 may determine that they are most interested in a business, for example because the name of the business (e.g., “Northwest Allergy and Immune Center”) seems to indicate a specialty in the overall issue the user 105 seems to be experiencing. The user 105 may request to view the complete information for the data card 715.21, resulting in generation of a new instance of the profile request data 507 and return of a new result data 320, including traversal of references of the business profile 130.21.

The profile traversal interface 704B presents another user interface focus, with the profile interface 718.21 presenting detailed information of the business profile 130.21. A visual 706 may be an image from an interior of one or more locations associated with the business profile 130, for example a photo of the inside of a primary room where tests and/or treatments related to the immune system are administered. Several points specified on the visual 706 may be clickable to open an expanded data card 716 related to a technology/product profile 140. In the present example, the user 105 may click a bottom point to open the expanded data card 716.24 (nested with the data card 715.21), which is a technology for immune system conditioning. This may be the most relevant option for the user 105, who may care more about finding out if their immune system can be adapted rather than changing their lifestyle. The user 105 may select a new option, “schedule an appointment”, which may initiate an action function 106 of the business profile 718.21 through the action response agent 322. Alternatively and/or in addition, the user 105 may select another action, “request more information”, which may trigger the action function 106 of the technology/product profile 140 which may email a brochure from the manufacturer and/or vendor of the immune conditioning technology to an email address entered by the user 105 and/or determined form the user profile 339 of the user 105. The user 105 may then end or continue their exploration. Each such selection may be utilized in adding new tags to the traversal criteria data 305, and/or adding an action request data 509 to a traversal data 416 usable for machine learning.

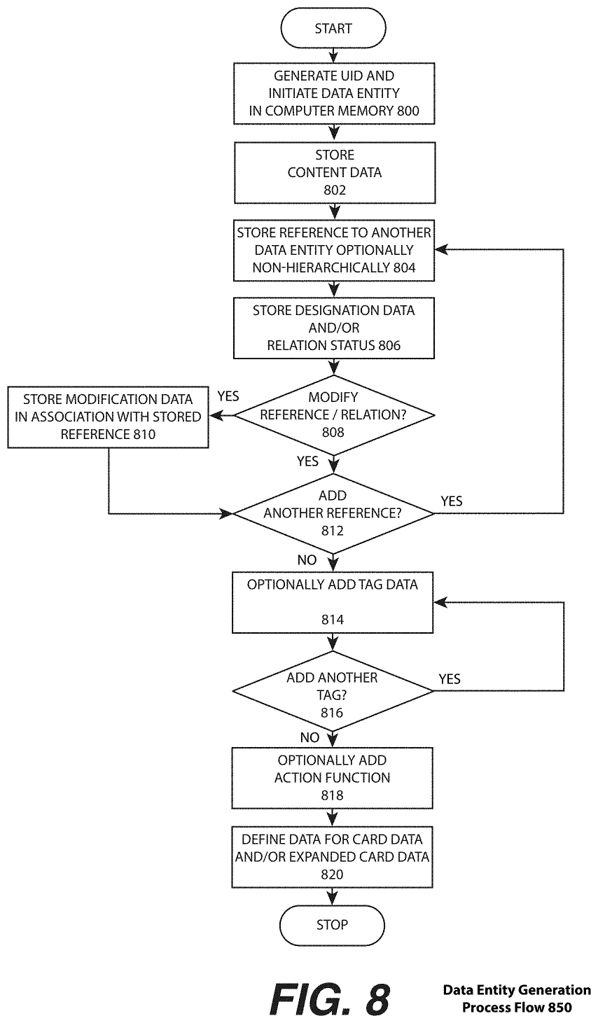

FIG. 8 is a data entity generation process flow 850, according to one or more embodiments. Operation 800 generates a unique identifier (e.g., the unique identifier 103) and initiates a data entity 101 in a computer memory (e.g., the memory 302 of FIG. 3). For example, the data entity 101.1 may be stored in one or more memory registers with one or more attributes initially storing undefined and/or null values. Operation 802 stores a content data 104 of the data entity 101, for example a name of the data entity 101 and/or a description of the data entity 101. Operation 804 stores a reference (e.g., the entity reference 112.1A) to another data entity 101 (e.g., the data entity 101.2). Operationally, the reference may be a non-hierarchical reference that is not constrained by definition of a hierarchical data structure, or other similar data structures with specified constraint such as a directed acyclic graph (DAG). Operation 804 may define the reference by storing a unique identifier 103 (e.g., the unique identifier 103.2) of the data entity 101 that is referenced in one or more reference attributes 110. Operation 806 stores a designation data 115 and/or a relation status 114 in association with the reference (e.g., the entity reference 112.1A). In one or more embodiments, the relation status 114 may apply to the entire data entity 101 (e.g., “unverified”), and/or may apply to one or more references (e.g., only to the entity reference 112.1A).

Operation 808 is a decision that determines whether the reference should be modified, for example to change its implied relationship. If the relation should be modified, operation 808 proceeds to operation 810, which stores a reference modification data 113 in association with the reference. The reference modification data 113, for example, may specify how to change, filter, or exclude any information derived from a referenced data entity 101 (e.g., the data entity 101.2). For example, the professional profile 120 may specify a service (e.g., diagnosis, treatment, or procedure) which they do not provide within those typically provided within a specialty. Operation 810 then proceeds to operation 812. If no modification of the relation is to occur, operation 808 may also proceed to operation 812.

Operation 812 determines if another reference is to be defined. If another reference is to be defined (e.g., an entity reference 112.1B), operation 812 returns to operation 804. Otherwise, all references within the reference attributes 110 may have been defined (e.g., all current semantic connections), and operation 812 may proceed to operation 814. Operation 814 may add a tag data 108 to the data entity 101. The tag data 108 may be directly stored in the data entity 101, and/or may be referenced (e.g., through a tag reference 109). Operation 816 is a decision determining whether another tag data 108 should be associated with the data entity 101. If another tag data 108 is to be associated, operation 816 returns to operation 814. Otherwise, operation 816 proceeds to operation 818. Operation 818 may add an action function (e.g., the action function 106) to the data entity 101. In one or more embodiments, the action function 106 may be selected from a set of predetermined functions, with a workflow for determining what action is triggered, any communication without outside systems and/or application programming interfaces (APIs), etc. In one or more embodiments, the action function 106 may be arbitrarily coded or otherwise defined by the user 107. Operation 820 may then define which data of the data entity 101, if any, may be included in a card data 214, an expanded card data 216, and/or the profile data 218. The data entity 101 may be stored in the semantic database 210.

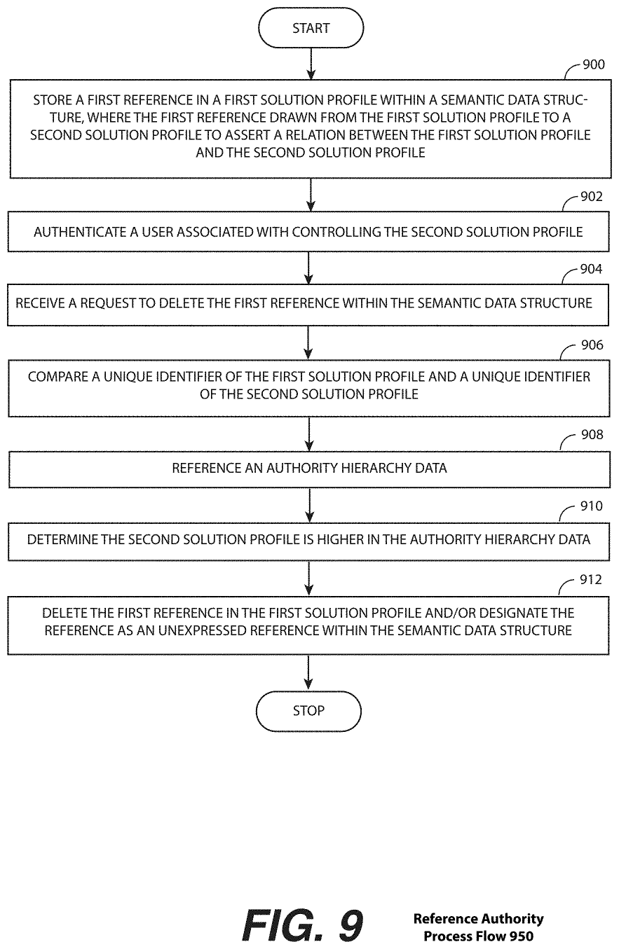

FIG. 9 is a reference authority process flow 950, according to one or more embodiments. Operation 900 stores a first reference (e.g., an entity reference 112.1A) in a first solution profile 102.1 within a semantic data structure 100. The first reference is drawn from the first solution profile 102.1 to a second solution profile 102.2 to assert a relation between the first solution profile 102.1 and the second solution profile 102.2. For example, the first solution profile 102.1 may be a business profile 130.1 and the second solution profile 102.2 may be a professional profile 120.2, where the reference (e.g., the entity reference 112.1A) asserts a relationship that the professional represented by the professional profile 120.2 works at the business modeled by the business profile 130.2. In one or more embodiments, both the solution profile 102.1 and the solution profile 102.2 may be initially defined and populated with pre-seeded data, for example obtained by parsing medical directories, sources of purchased information, scraping available online sources, etc. Operation 902 authenticates a user 107 (e.g., the user 107B of FIG. 1B) associated with controlling the second solution profile 102.2. For example, the user 107 may have claimed the right to own and/or control the solution profile 102.2. Operation 904 receives a request to delete the first reference 112.1A within the semantic data structure 100, for example from the user 107 controlling the second solution profile 102.2.

Operation 906 compares a unique identifier 103 of the first solution profile 102.1 (e.g., the unique identifier 103.1) and a unique identifier 103 of the second user profile 102.2 (e.g., the unique identifier 103.2). The type of each data entity 101 and/or solution profile 102 may also be determined. Operation 908 may then reference an authority hierarchy data 321. The authority hierarchy data 321 may, for example, define a set of rules and/or conditions for one data entity 101 and/or solution profile 102 to have authority over another solution profile 102. For example, a professional profile 120.2 may have authority to change and/or delete a reference (e.g., the entity reference 112.1A) to that professional profile 120.2 stored in the business profile 130.1 in all cases, or, alternatively, as long as the business profile 130.1 is unverified. Operation 910 may determine the second solution profile 102.2 is higher in the authority hierarchy data 321 (including any additional data or factors related to both the first solution profile 102.1 and the second solution profile 102.2 relevant to the determination). Operation 912 may then delete the first reference (e.g., the entity reference 112.1A) in the first solution profile 102.1 and/or designate the reference as an unexpressed reference (e.g., the unexpressed reference 117) within the semantic data structure 100 (as may be designated in either the first solution profile 102.1 and/or the second solution profile 102.2 such as by specifying non-expression in a designation data 115).

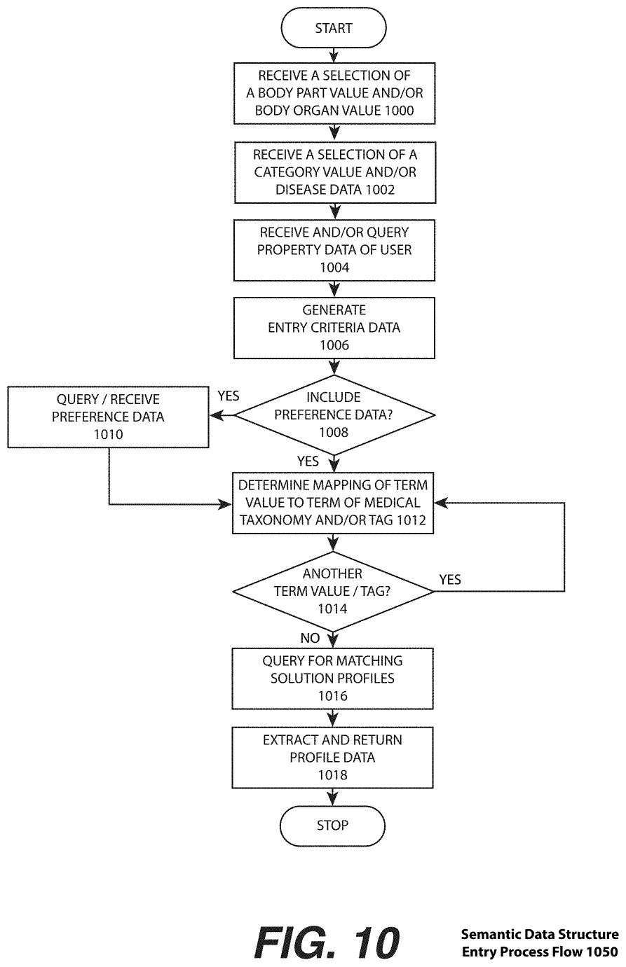

FIG. 10 is a semantic data structure entry process flow 1050, according to one or more embodiments. Operation 1000 receives a selection of a body part value 602 and/or a body organ value 604. In one or more embodiments, multiple values of each may be selected, for example “shoulder” and “neck”, or “lungs” and “throat”. Operation 1002 receives a selection of a category value (e.g., a general category value 606 and/or a specific category value 608) and/or a disease value 610. Operation 1004 receives and/or queries a property data 612 of the user 105. For example, the property data 612 may be received from manual entry by the user 105 through a workflow and/or may be extracted from a user profile 339 of the user 105. Operation 1006 then may generate an entry criteria data 505 comprising one or more pieces of data specified in operation 1000 through operation 1004. The entry criteria data 505 may be generated on the client device 500 and/or the coordination server 300.

Operation 1008 is a decision determining whether a set of preference data 335 should be considered in applying filters in determination of the entry nodes of the semantic data structure 100 and/or during traversal of the references of the semantic data structure 100. Where preference data 335 is to be utilized, operation 1008 proceeds to operation 1010 which may receive a selection (e.g., manually from the user 105 as part of the workflow) and/or extract the preference data 335 from the user profile 339 associated with the user 105. Operation 1012 may determine a mapping from a first term value in an entry criteria data 505 to a second terminology value 222 of a medical taxonomy 224. In one or more embodiments, operation 1012 determines a tag data 108 that matches the entry criteria data 505. The tag data 108 may be mapped to a terminology value 222 of a medical taxonomy 224. Operation 1014 determines whether another tag data 108 applies. If another tag data 108 applies, operation 1014 returns to operation 1012. Otherwise, operation 1014 proceeds to operation 1016. Operation 1016 queries one or more solution profiles 102 matching the terminology value 222 and/or tag data 108. For example, the solution profiles 102 may match because they also include a reference to the tag data 108, and/or because they includes within their content data 104 or other data the terminology value 222 to which the tag data 108 is mapped. Operation 1018 may then extract and return profile data, for example the profile data 218 and/or the card data 214 of the solution profile 102 (e.g., within the result data 230). Note that in the embodiment of FIG. 10, filtering may also be carried out as described throughout the present embodiments, but is not shown (e.g., an operation 1017 following the operation 1016 and prior to the operation 1118).

FIG. 11 is a semantic data structure traversal process flow 1150, according to one or more embodiments. Operation 1100 receives a solution profile request (e.g., the profile request data 507 for retrieval of data a solution profile 102. The solution profile request may include an identifier and/or a unique identifier 103 of the solution profile 102. Operation 1102 queries the solution profile 102 (and/or a data entity 101). For example, the query agent 204 may receive the unique identifier 103 of the solution profile and address it within the semantic database 210. Operation 1104 optionally determines a tag data 108 of the solution profile 102 and adds the tag data 108 to the traversal criteria data 305. In one or more embodiments, because the solution profile 102 was requested one or more tag data 108 may be relevant to continued navigation and/or traversal of the semantic database 210.

Operation 1106 identifies and traverses a reference (e.g., an entity reference 112) of the solution profile 102 and stores a unique identifier 103 of the data entity 101 to which the reference is drawn. Operation 1108 determines whether data entity 101 is relevant to the user 105, for example based on the entry criteria data 505, the traversal criteria data 305, and/or the profile request data 507. In one or more embodiments, the relevance may be determined through determining a match between a tag data 108 stored in the traversal criteria data 305 and a tag data 108 associated with the data entity 101. If relevance is not initially determined, operation 1108 may proceed to operation 1109 which is a decision which may be used to determine whether relevance should be expanded. In one or more embodiments, the expansion may occur randomly (e.g., to introduce randomness into the search), and/or may be introduced when not enough unique identifiers 103 are stored (e.g., in the traversal set 212A and/or the traversal set 212B of FIG. 3). Alternatively, or in addition, expansion of results returned in the result data 320 may occur through limiting filters and/or preferences (e.g., reducing limiting factors of the preference data 335). Where no expansion of traversal and/or inclusion is to occur, operation 1109 proceeds to operation 1111 which discards the unique identifier 103 of the data entity 101 and proceeds to operation 1112.

If either operation 1108 determines the data entity 101 is relevant or operation 1109 determines matching should be expanded to include the data entity 101, the respective operations proceed to operation 1110 which stores the unique identifier 103 in a set of unique identifiers (e.g., the traversal set 212). Operation 1112 is a decision determining whether another reference is stored in the data entity 101. For example, the set of reference attributes 110 may be sequentially assessed until all references have been assessed. If additional references are untraversed, operation 1112 may return to operation 1106 to be applied to identify and traverse the next reference. Otherwise, operation 1112 may proceed to operation 1114.

Operation 1114 extracts a profile data 218 for the solution profile 102, and may extract a card data 214 for each unique identifier 103 within the set of solution profiles 102 within the traversal set 212. Prior to operation 1114, the traversal set 212 may be filtered (e.g., filtered by the filter subroutine 314 of FIG. 3). In addition, any portion of the profile data 218 which may rely on a derivative reference 116 may also be extracted (e.g., the locations associated with a business where a professional works, as shown and described in conjunction with the embodiment of FIG. 1D and FIG. 1E). Operation 1116 may then assemble and return the result data 230.

Although not shown in the embodiment of FIG. 11 for ease of illustration, each data entity 101 to which a reference is drawn may also become the subject of a branching traversal. For example, an operation 1115 (not shown) between operation 1114 and operation 1116 may determine whether sufficient unique identifiers 103 are stored in the traversal set 212 and, if below a threshold requirement, initiate another branching beginning with querying one of the unique identifiers 103 within the traversal set 212 in operation 1102 (e.g., the data entity 101 would be begin the process flow of FIG. 11 in operation 1102). Additionally, although a solution profile 102 is described in FIG. 11 as requested and queried, it will be recognized that a data entity 101 that is not a solution profile 102 may be similarly requested and the semantic data structure 100 traversed according to the embodiment of FIG. 11.

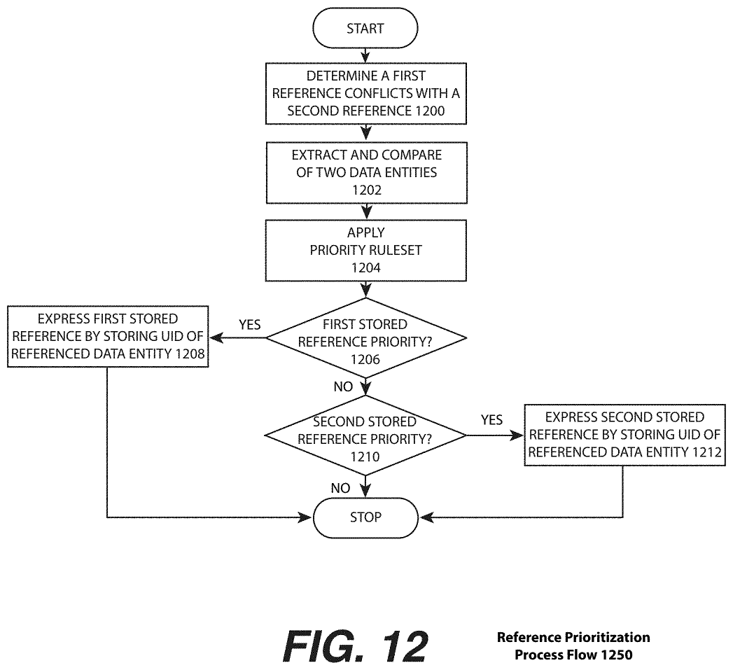

FIG. 12 is a reference prioritization process flow 1250, according to one or more embodiments. In operation 1200 a first stored reference (e.g., a entity reference 112.1 of a data entity 101.1) is determined to conflict with a second stored reference (e.g., an entity reference 112.2 of a data entity 101.2). For example, the conflict may be determined due to an assertion of a first solution profile 102.1 with an assertion by a second solution profile 102.1, where both assertions are mutually exclusive. In just one example, it may be unusual and unlikely for a business profile 130 to have each of five models of MRI machine, many of which have overlapping function, and therefore each entity reference 112 may have to be analyzed before expression or, in the absence of verification (e.g., as may be stored as and/or recorded in a profile verification data), one of the references determined to be prioritized for expression. In another example, two clinics were entered with the same address but different profiles. Alternatively, or in addition, operation 1200 may determine that a stored reference makes and/or implies an assertion and/or a relation that must be assessed before expression. For example, a new profile of a doctor (e.g., a professional profile 120B) is created, which only differs from an existing profile (e.g., a professional profile 120A) in that one lists a middle name and the other does not. Operation 1202 extracts and compares data of two or more data entities 101 that may be analyzed to determine priority. In one or more embodiments, a relation status 114 of an entity reference 112 may be extracted, where the relation status 114 is: (i) a profile unverified status designating that the first data entity 101.1 is unassociated with a user profile 339, (ii) a relation unverified status designating that the first entity reference 112 is unreviewed by a user profile 339 referenced as a controlling user profile 339 of the first data entity 101, (iii) a relation pending status designating that the first entity reference 112 is to be verified by the user profile 339 referenced as controlling the first data entity 101 and/or a user profile 339 referenced as controlling the second data entity 101.2, (iv) a profile verified status designating that the first data entity 101.1 is controlled by the user profile 339, and (v) a relation verified status designating that the first entity reference 112 has been verified by the user profile 339 referenced as controlling the first data entity 101.1. Other data what may be usable to determine priority can include, for example, metadata about the solution profile 102 (e.g., how long it has been associated with a controlling user profile 339, how active the controlling user profile 339 has been, etc.).

Operation 1204 applies a priority ruleset. The priority ruleset, for example, may specify comprise computer readable instructions that accepts a set of input data from the first data entity 101.1 and the second data entity 101.2 (and/or the first entity reference 112.1A and the second entity reference 112.1B of the same data entity 101.1) and outputs whether the first stored reference or the second stored reference should be expressed. In one or more embodiments the priority ruleset may be based on a decision tree, a points system, weighted factors, and/or other means of determining a priority of the first stored reference versus the second stored reference. In one or more embodiments, the priority ruleset may also utilize the authority hierarchy data 321 to determine expression priority.

Operation 1206 determines whether the first stored reference receives priority. If the first stored reference is to receive priority, operation 1206 proceeds to operation 1208 which expresses the first stored reference by storing the unique identifier 103 of the data entity 101 referenced by the first stored reference (and/or utilizing the first stored reference to lead to a derivative reference 116). If the first stored reference is not to be expressed, operation 1206 proceeds to operation 1210, which determines if the second stored reference should be expressed. If the second stored reference is to be expressed, operation proceeds to operation 1212 which expresses the second stored reference by storing the unique identifier of the data entity 101 referenced by the second stored referenced. Otherwise, operation 1210 may proceed to end, resulting in both the first stored reference and the second stored reference being unexpressed.

FIG. 13 is a reference save and compare process flow 1350, according to one or more embodiments. Operation 1300 selects a solution profile 102 to save in association with a user profile 339. The selection may be made by the user 105 on a user interface, for example by clicking a “save” profile button on a data card 715. Operation 1302 may traverse the incoming and/or outgoing references of the solution profile 102 at a first time to determine the context and/or current relations of the solution profile 102. Operation 1204 stores a reference snapshot data 325 at the first time (e.g., a reference snapshot data 325A), which may include the unique identifiers 103 of data entities 101 which the solution profile 102 references and/or is referenced by. Operation 1206 may then store a reference to the solution profile 102 (e.g., the entity reference 112 shown within the user profile 339 in FIG. 3) and in association store the reference snapshot data 325.

Operation 1208 optionally subscribes to the solution profile 102, such that the user 105 may be notified of a change in data of the solution profile 102 (e.g., the content data 104, an entity reference 112). Similarly, operation 1210 generates a notification data that includes the change and transmits the notification data to the client device 500 to the user 105 (e.g., through email, through an app on the client device 500, etc.).

Operation 1212 receives a profile request data 507 for the saved solution profile 102 from the client device 500, for example at a second time (e.g., 3 months later). Operation 1214 re-traverses the semantic data structure 100 such as the incoming and/or outgoing entity references 112 of the solution profile 102, which may have changed between the first time and the second time (e.g., a clinician may have moved clinics, or a business may have acquired a new technology at one of their locations). Operation 1216 optionally compares the semantic connections (e.g., the incoming and/or outgoing references) at the first time and the second time by comparing the reference snapshot data 325A at the first time with a new reference snapshot data 325B generated at the second time. Any differences may then be returned to the user 105 on the client device 500 (e.g., as the variation data 327), including in easily readable formats such as side-by-side data cards 715 at the first time and the second time, a report of the changes, a marked-up data card 715, etc.

FIG. 14 is a machine learning process flow 1450, according to one or more embodiments. Operation 1400 initiates a training session. For example, the training session may be initiated when a user 105 logs in (e.g., authenticates), begins a new workflow for generation of the entry criteria data 505, and/or at an arbitrary time during traversal of the semantic data structure 100. Operation 1402 extract a set of unique identifiers 103 of at least one of a set of data cards 715 expanded by the user 105 (e.g., a unique identifier 103 of the solution profile 102 associated with the displayed data card 715), a set of solution profiles 102 having profile data 218 requested by the user 105, a set of stored references from the user profile 339 to one or more solution profiles 102 (e.g., a saved instance of the solution profile 102). Each of such interactions may be referred to as an interaction function 418, which may be stored in sequence, with timestamps, and/or with other metadata describing how each was generated. The set of unique identifiers 103 and each associated interaction function 418 may be stored as a traversal data 416, as shown in the embodiment of FIG. 4. The traversal data 416 may continue to grow (receive pairs of the unique identifier 103 and the interaction function 418) until a condition occurs, for example receipt of an action request data 509 is determined.

Operation 1404 determines occurrence of a profile action (e.g., through receipt of an action request data 509) of the user 105 associated with a solution profile 102, for example generation of an action request data 509 for triggering an action function 106 of the solution profile 102. Operation 1406 then extracts the unique identifier 103 of the solution profile 102. The action request data 509 and an associated unique identifier 103 of the solution profile 102 may be added to the traversal data 416. In one or more embodiments, determination of the profile action may also modify the traversal data 416, for example limiting the traversal data 416 to the last eight major interactions of the semantic data structure 100.

Operation 1408 input as a precursor path 420 (e.g., the first precursor path 420.1 of FIG. 4) comprising a set of unique identifiers 103 as one or more inputs into an artificial neural network 404. In one or more embodiments, the artificial neural network 404 includes two or more processing nodes 406 each comprising one or more weighted outputs 408. For example, the precursor path 420 may be input such that each unique identifier 103 has an input into a distinct processing node 406. Operation 1410 inputs as an applicable selection 422 the unique identifier 103 of the solution profile 102 (e.g., for which the action request was generated) and a tag data 108 of the solution profile 102. Operation 1412 may then adjust an output weight of one or more of the one or more weighted outputs 408 to train the artificial neural network 404. It should be noted that initial training of the artificial neural network 404 may be accomplished through large data training sets as may be known in the art, and/or the training may occur asynchronously from the traversal of the semantic data structure 100.

FIG. 15 is a machine learning predication process flow 1550, according to one or more embodiments. Operation 1500 may extract a set of unique identifiers 103 of a set of data cards 715 expanded by the user 105, a set of solution profiles 102 having profile data 218 requested by the user 105, and/or a set of stored references from the user profile 339 to one or more solution profiles 102. Such interactions the user 105 has with the semantic database 210 and one or more of its data entities 101 may be stored as a traversal data 416. Relative to operation 1402 of FIG. 14, the extraction may be of a different set of data cards 715 expanded by the user 105, a different set of solution profiles 102 having profile data 218 requested by the user 105, and/or a different set of stored references from the user profile 339 to one or more different solution profiles 102.

Operation 1502 inputs as a second precursor path 420 (e.g., the precursor path 420.2 of FIG. 4) a set of unique identifiers 103 as one or more inputs into the artificial neural network 404 comprising the two or more processing nodes 406 each comprising the one or more weighted outputs 408. Operation 1504 outputs from the artificial neural network 404 a tag data 108 and/or the unique identifier 103 of a solution profile 102 (e.g., as the output 423). The unique identifier 103 of the solution profile 102 within the output 423 may be an applicable selection 424 predicted to be applicable and/or relevant to the user 105. Similarly, the tag data 108 may be an applicable selection 424 predicted to be applicable and/or relevant to continued search, navigation, and traversal by the user 105. It should be noted that one or more sets of the traversal data 416 may overlap, including forming overlapping precursor paths 420, each of which may generate a distinct output 423.

Operation 1506 query the solution profile 102 with the unique identifier 103 of the solution profile 102 and/or a solution profile 102 referencing the tag data 108 (e.g., after adding the tag data 108 to the traversal criteria data 305 and performing one or more additional queries). Operation 1508 transmits the card data 214 of the solution profile 102 referenced in the output 423 and/or the solution profile 102 referenced by the tag data 108 to the client device 500 of the user 105.

With respect to the predicted applicable selection 424, if the user 105 generates an action request data 509 to initiate an action function 106 of the solution profile 102, the user 105 may, according to one or more embodiments, have demonstrated that the prediction was correct. In such case, the traversal data 416 used to generate the output 423 may be also used to train the artificial neural network 404 further, and/or techniques known in the art may be applied to confirm the correctness (or lack of correctness) of the prediction, for example neural network error back-propagation (e.g., “backprop”).

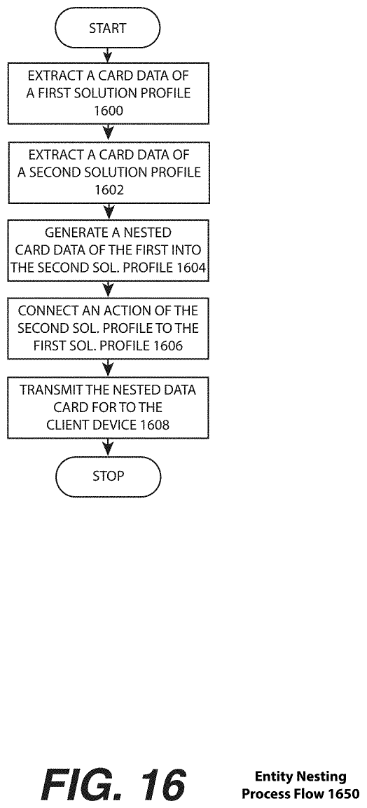

FIG. 16 is an entity nesting process flow 1650, according to one or more embodiments. Operation 1600 extracts a card data 214 of a first solution profile 102.1. Operation 1602 extracts a card data 214 of a second solution profile 102.2. For example, operation 1600 may extract the card data 214 of a professional profile 120, and operation 1602 may extract a card data 214 of a service profile 150. Operation 1602 generates a nested data card of the first solution profile 102.1 placed into the second solution profile 102.2. for example, the nested card data may have the title of the service profile 150, a subtitle of the professional profile 120, and a location (e.g., the name of the city) of a business profile 130 to which the professional profile 120 references (e.g., delivered through a business reference 132 of the professional profile 120). Operation 1606 connects an action function 106 of the second solution profile 102 to the first solution profile 102. For example, a button with “schedule an appointment” may trigger the action function 106 of the professional profile 120. Operation 1608 then transmits the nested card data to the client device 500 (e.g., for display as a nested data card 720, as shown and described in conjunction with the embodiment of FIG. 7).

A set of example graphical outputs illustrating user experience in entering, traversing, and interacting with data entities 101 of the semantic data structure 100 is now provided. The additional underling operations, including entry and traversal of the semantic data structure, are shown and described in FIG. 1A through FIG. 16, and throughout the present embodiments.

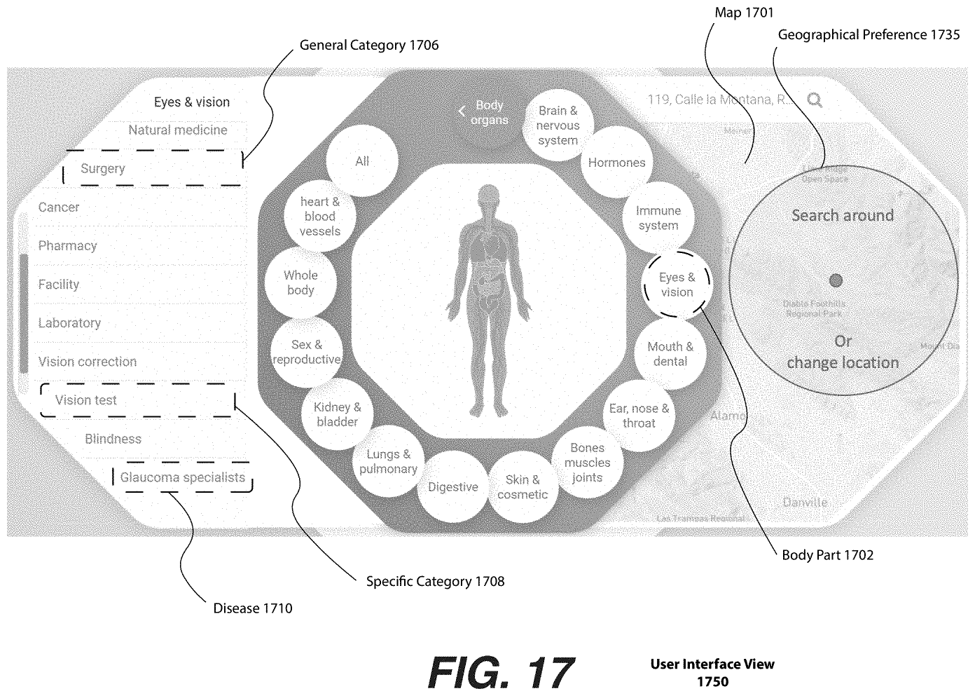

FIG. 17 illustrates an example user interface view 1750 of a workflow available to the user 105, for example on the client device 500, for selecting body part value 602, a body organ value 604, a disease value 610, and/or a category value (e.g., the general category value 606, the specific category value 608), usable for determining one or more initial entry nodes into the semantic data structure 100, according to one or more embodiments.

In one or more embodiments and the embodiment of FIG. 17, the user 105 is provided a workflow on a graphical user interface for easily selecting an initial set of data for determining entry nodes into the semantic data structure 100. In the present example, the user 105 may be attempting to find out if their child may a new glasses prescription, for example because the child has begun exhibited new learning difficulties in school. First, the user 105 may select a body part and/or a body organ item form a menu (e.g., the body part 1702).

In the present embodiment, the user 105 may select “Eye and Vision”. Selection of the body part 1702 on the graphical user interface may add a corresponding instance of the body part value 602 to the entry criteria data 505. Next, the user 105 may select a category or disease, for example either the general category 1706 (e.g., “surgery”), the specific category 1708 (e.g., “vision test”), or the disease 1710 (e.g., “glaucoma”) which may add, respectively, a general category value 606, a specific category value 608, or a disease value 610 to the entry criteria data 505. In the present embodiment, the user 105 may select “vision test,” which in one or more embodiments may be an instance of the specific category 1708. Also illustrated, the user 105 may select one or more preferences. As shown in the embodiment of FIG. 17, a map 1701 includes a movable radius providing the user 105 the ability to provide a geographic preference 1735 (e.g., as a means to select a portion of the preference data 335).

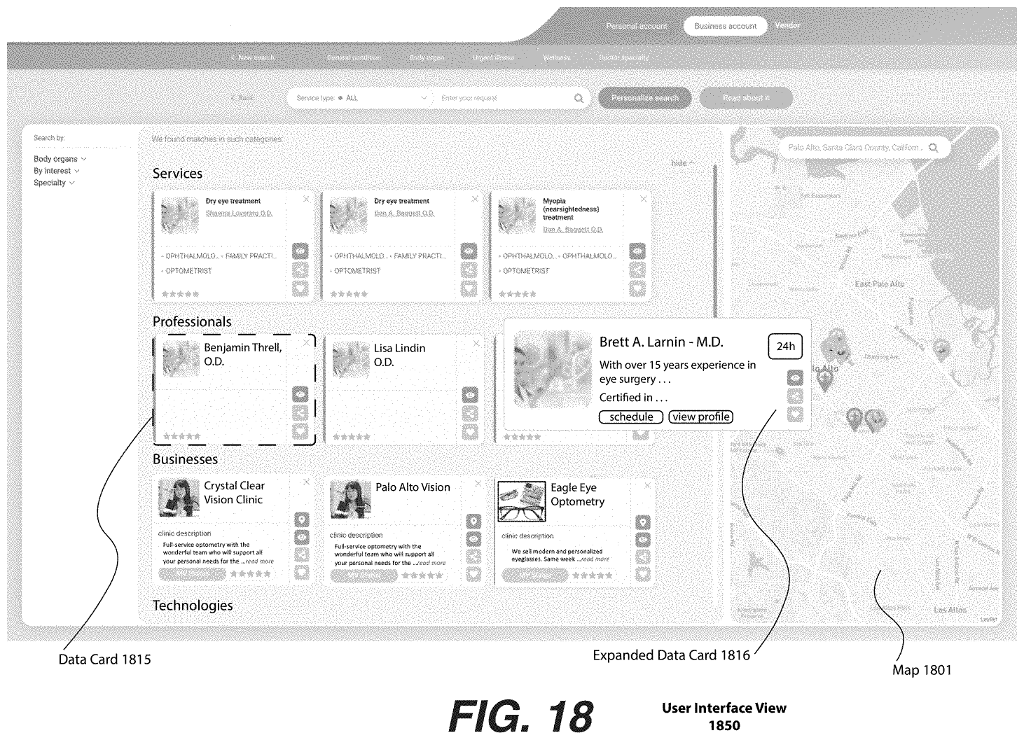

FIG. 18 illustrates another example user interface view 1850 illustrating a set of data cards 715 generated from card data 214 extracted from a set of solution profiles 102 following initial entry into one or more nodes of the semantic data structure 100 as shown in FIG. 17, according to one or more embodiments. FIG. 18 illustrates a set of categorized data cards 715 from solution profiles 102, including three instances of the data card 715 generated from card data 214 extracted from service profiles 150, three instances of the data card 715 generated from card data 214 extracted from professional profiles 120, and three instances of the data card 715 generated from card data 214 extracted from business profiles 130. A map 1801 illustrates locations associated with one or more of the set of data cards 715. One of the instances of the data card 715 is expanded, labeled as the expanded data card 1816. The expanded data card 1816 illustrates two action buttons (e.g., “schedule” and “view profile”), along with a descriptor of responsiveness (“24 hours”) that may have been defined by the user 107 associated with the professional profile 120 from which the expanded data card 1816 was derived. Following review of multiple options for entering the semantic data structure 100 to begin traversal, the user 105 may select to view an instance of the data card 715 labeled the data card 1815 in the present embodiment for “Benjamin Threll, O.D.”. The unique identifier 103 of the professional profile 120 corresponding to the data card 1815 may be submitted within a profile request data 507.

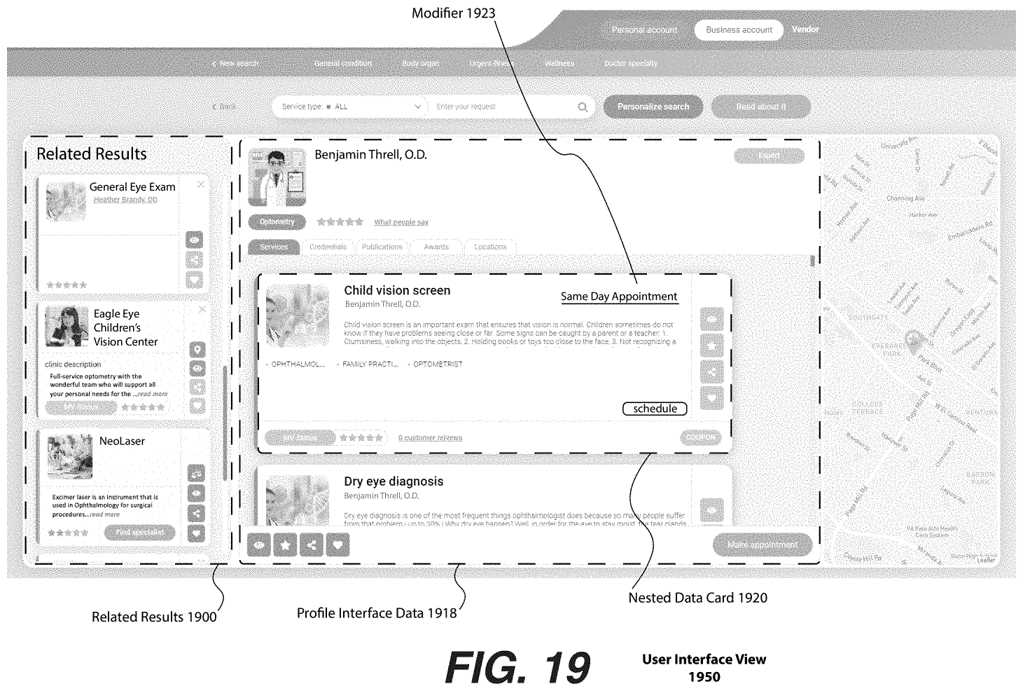

FIG. 19 illustrates yet another example user interface view 1950 illustrating a user interface displaying a profile data 218 of a professional profile 120 as a main focus of the user interface (e.g., as was selected from the data card 1815 of FIG. 18), according to one or more embodiments. FIG. 19 additionally illustrates three data cards 715 of solution profiles 102 referenced by and/or drawing references to the professional profile 120 that is the main focus (e.g., labeled the related results 1900), according to one or more embodiments. The profile data 218 of the professional profile 120 is illustrated in a central area of the user interface, including dividing information into several tabs for efficient navigation by the user 105. (e.g., “services,” “credentials,” “publications”, “awards”, and “locations.”). The “services” tab shown actively selected additionally may present a set of nested data cards 720 listing services provided by the professional (e.g., as may be derivatively referenced through the specialty reference 172.1 and one or more service references 152.6 as shown and described in conjunction with FIG. 1D). In particular, the nested data card 1920 is shown to include a modifier 1923 specifying that same-day service is available, labeled as the modifier 1923. The modifier 1923 may modify the specialty reference 172 and/or the service reference 152, for example stored as the reference modification data 123.1 as shown and described in conjunction with the embodiment of FIG. 1D. The related results 1900 may illustrate one or more solutions profiles 102 related to the professional profile 120 of Benjamin Threll, O.D., as shown and described throughout the present embodiments. After reviewing the profile of Benjamin Threll, O.D. and noticing there are child-specific services related to eyes and vision (e.g., “Child vision screen”), the user 105 may decide they are interested in a location that specializes in children's vision care. The user 105 may then notice a related result presented as a data card 715 for “Eagle Eye Children's” vision center within the related results 1900.

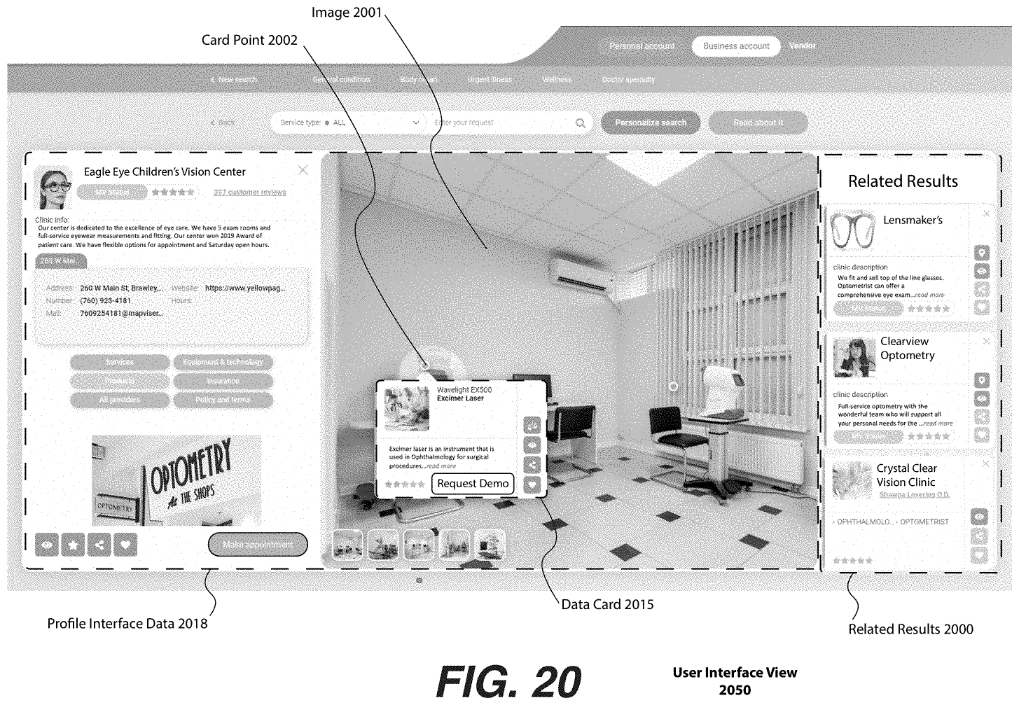

FIG. 20 illustrates yet another example of a user interface view 2050 illustrating a profile data 218 of a business profile 130 selected from a data card of FIG. 19 as a main focus (e.g., the profile interface data 2018), according to one or more embodiments. FIG. 20 additionally illustrations three more instances of the data cards 715 of solution profiles 102 referenced by and/or drawing references to the business profile 130 that is the main focus (e.g., the related results 2000). Additionally, an image 2001 associated with the business profile 130 is presented with a number of selectable “card points” (e.g., the card point 2002). Each card point 2002 may be expandable into a data card 715, which may in turn be expandable into an expanded data card 716. The image 2001 may permit the user 105 an accessible visual transition to assist in understanding the relationship between a business and products, services, and/or technology, including assisting the user 105 in traversing the semantic data structure 100 to new solution profiles 102. In the present example, the user 105 may see an interesting piece of equipment in the image 2001 represented by the labeled instance of the card point 2002 and click resulting in expansion of the card data 2015. In the present example, the user 105 may be very interested to find that surgical procedures for vision correction may be available for children, and may therefore want to learn more so they can ask about the technology during the appointment. The user 105 may click “request demo” as an action button of the data card 715 (e.g., defined in data of the card data 214), which may initiate an action function 106 of a technology/product profile 140.3, as shown and described in FIG. 1D.

Although the present embodiments have been described with reference to specific example embodiments, it will be evident that various modifications and changes may be made to these embodiments without departing from the broader spirit and scope of the various embodiments. For example, the various devices, engines, agent, routines, and modules described herein may be enabled and operated using hardware circuitry (e.g., CMOS based logic circuitry), firmware, software, or any combination of hardware, firmware, and software (e.g., embodied in a non-transitory machine-readable medium). For example, the various electrical structure and methods may be embodied using transistors, logic gates, and electrical circuits (e.g., application specific integrated circuitry (ASIC) and/or Digital Signal Processor (DSP) circuitry).

In addition, it will be appreciated that the various operations, processes, and methods disclosed herein may be embodied in a non-transitory machine-readable medium and/or a machine-accessible medium compatible with a data processing system (e.g., the database server 200, the coordination server 300, the machine learning server 400, the client device 500, and/or the artificial neural network 404). Accordingly, the specification and drawings are to be regarded in an illustrative rather than a restrictive sense.

The structures in the figures such as the engines, routines, and modules may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the preceding disclosure.

Embodiments of the invention are discussed above with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems.

Features which are described in the context of separate embodiments may also be provided in combination in a single embodiment. Conversely, various features which are, for brevity, described in the context of a single embodiment, may also be provided separately or in any suitable sub-combination. The applicants hereby give notice that new claims may be formulated to such features and/or combinations of such features during the prosecution of the present application or of any further application derived therefrom.

References to “one embodiment,” “an embodiment,” “example embodiment,” “various embodiments,” “one or more embodiments,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least one or more embodiments of the invention” includes the stated particular feature, structure, or characteristic.

The enumerated listing of items does not imply that any or all of the items are mutually exclusive, unless expressly specified otherwise.

It is understood that the use of a specific component, device and/or parameter names are for example only and not meant to imply any limitations on the invention. The invention may thus be implemented with different nomenclature and/or terminology utilized to describe the mechanisms, units, structures, components, devices, parameters and/or elements herein, without limitation. Each term utilized herein is to be given its broadest interpretation given the context in which that term is utilized.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; a smartphone, application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, one or more embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The term “processor” may refer to any device or portion of a device that processes electronic data from registers and/or memory to transform that electronic data into other electronic data that may be stored in registers and/or memory. A “computing platform” may comprise one or more processors.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the semantic data structure system 194 according to the present invention will be apparent to those skilled in the art. Various aspects of the invention have been described above by way of illustration, and the specific embodiments disclosed are not intended to limit the invention to the particular forms disclosed. The particular implementation of the loyalty rewards programs may vary depending upon the particular context or application. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

We claim:

1. A method for efficient determination of one or more data entities in a database storing information applicable to a user, the method comprising:
generating an entry criteria data for calculation of an entry point into one or more nodes of a semantic data structure, the entry criteria data comprising:
at least one of a body part value and a body organ value, and
at least one of a general category value, a specific category value that is specific to at least one of the body part value and the body organ value, and a disease value, and
receiving a preference data of the user;
querying the semantic data structure to receive a first set of unique identifiers of a plurality of data entities each comprising a tag data matching the entry criteria data,
wherein the tag data matching the entry criteria data is mapped from a term value of a medical taxonomy through a tag reference to the tag data, and
wherein the plurality of data entities comprising solution profiles comprising at least one of:
a service profile,
a professional profile, and
a technology profile;
removing a first subset of the first set of unique identifiers based on at least one of a location data associated with the user stored in the user profile and the preference data of the user to result in a second set of unique identifiers; and
transmitting to a client device of the user a card data for each data entity associated with the second set of unique identifiers.

2. The method of claim 1, further comprising:
receiving a first profile request for a first solution profile comprising a unique identifier of the first solution profile; and
transmitting to the client device of the user a profile data of the first solution profile.

3. The method of claim 2, further comprising:
traversing within the semantic data structure one or more references of the first solution profile to a set of solution profiles;
storing a third set of unique identifiers comprising the set of solution profiles referenced through the one or more references of the first solution profile upon receiving the first profile request;
removing a second subset of unique identifiers from the third set of unique identifiers based on the location data associated with the user stored in the user profile and the preference data of the user to result in a fourth set of unique identifiers; and
transmitting to the client device of the user a card data for each solution profile associated with the fourth set of unique identifiers to present semantically related data entities traversable by the user within the semantic data structure to increase a probability of an efficient connection between the solution profile that is applicable to the user.

4. The method of claim 3, further comprising:
initiating a traversal criteria data initially comprising the entry criteria data;
extracting a tag data of the first solution profile upon receiving the first profile request; and
storing the tag data of the first solution profile in the traversal criteria data,
wherein the third set of unique identifiers further comprising a second set of solutions profiles referencing the tag data of the first solution profile.

5. The method of claim 4, further comprising:
determining that a stored reference within in the semantic data structure as an attribute of a second solution profile of the set of solution profiles is a unidirectional reference to a third solution profile;
determining the stored reference is prioritized based on at least one of a profile verification data, an entity type data, an entity size data, and a timestamp of the stored reference;
determining the stored reference of the second solution profile is an expressed reference,
wherein a unique identifier of the second solution profile is included in the third set of unique identifiers; and
transmitting to the client device of the user a card data of the third solution profile to increase a probability of modeling accuracy through expression of the stored reference as the expressed reference.

6. The method of claim 5, further comprising:
storing a reference from the user profile to a unique identifier of the first solution profile to enable the user to re-query the first solution profile;
receiving a second profile request for the solution profile comprising a unique identifier of the solution profile; and
transmitting to the client device of the user at least one of the card data of the first solution profile and the profile data of the first solution profile upon receiving the second profile request;
re-traversing, upon receiving the second profile request, one or more references of the first solution profile;
storing a fifth set of unique identifiers;
transmitting to the client device of the user a card data for each data entity associated with the fifth set of unique identifiers to present re-calculated semantically related data entities traversable by the user.

7. The method of claim 6, further comprising:
generating at a first time a first reference snapshot data of one or more references referencing a solution profile saved by the user, the first reference snapshot data comprising a unique identifier one or more data entities referencing the solution profile;
generating at a second time a second reference snapshot data of one or more references referencing the solution profile saved by the user, the second reference snapshot data comprising a unique identifier one or more data entities referencing the solution profile;
determining that the first profile data at the first time and the first profile data at the second time vary; and
transmitting a variation data to the user.

8. The method of claim 7, further comprising:
initiating a training session;
extracting a sixth set of unique identifiers of a set of solution profiles that have at least one of a set of data cards expanded by the user, a profile data requested by the user, and a set of stored references from the user profile one or more solution profiles;
determining receipt of an action request of the user associated with a fourth solution profile;
extracting the unique identifier of the fourth solution profile;
inputting as a first precursor path comprising the sixth set of unique identifiers as a first set of one or more inputs into an artificial neural network comprising two or more processing nodes each comprising one or more weighted outputs;
inputting as an applicable selection at least one of the unique identifier of the solution profile and a tag data of the fourth solution profile; and
adjusting an output weight of one or more of the one or more weighted outputs to train the artificial neural network.

9. The method of claim 8, further comprising:
extracting a seventh set of unique identifiers of a set of solution profiles that have at least one of a different set of data cards expanded by the user, a different profile data requested by the user, and a different set of stored references from the user profile drawn to one or more solution profiles;
inputting as a second precursor path the seventh set of unique identifiers as a second set of one or more inputs into the artificial neural network comprising the two or more processing nodes each comprising the one or more weighted outputs;
outputting from the artificial neural network at least one of a second tag data and the unique identifier of a fifth solution profile;
querying at least one of: (i) the fifth solution profile with the unique identifier of the fifth solution profile and (ii) a sixth solution profile referencing the second tag data; and
transmitting a card data of at least one of the fifth solution profile and the sixth solution profile to the client device of the user.

10. The method of claim 9, further comprising:
storing a first reference in the first solution profile within the semantic data structure, the first reference drawn from the first solution profile to the second solution profile to assert a relation between the first solution profile and the second solution profile;
authenticating a second user controlling the second solution profile;
receiving a request to delete the first reference within the semantic data structure;
comparing a unique identifier of the first solution profile and a unique identifier of the second solution profile;
referencing an authority hierarchy data;
determining the second solution profile is higher in the authority hierarchy data; and
at least one of (i) deleting the first reference in the first solution profile and (ii) designating the first reference as an unexpressed reference within the semantic data structure.

11. The method of claim 10, further comprising:
extracting a card data of the first solution profile;
extracting a card data of the second solution profile;
generating a nested card data that incorporates at least a portion of the card data of the first solution profile into the card data of the second solution profile;
transmitting the nested card data for the second solution profile to the client device of the user;
reading a relation status value from the first solution profile;
transmitting the relation status value within the card data of the first solution profile for display on the client device of the user to communicate an association strength within the semantic data structure of the first solution profile to the second solution profile;
mapping the tag data matching the entry criteria data comprising at least one of the body part value, the body organ value, the general category value, the specific category value, and the disease value, to a term value of the medical taxonomy through the tag reference to the tag data;
receiving a third profile request for an expanded card, the third profile request comprising a unique identifier of a seventh solution profile;
transmitting to the client device of the user an expanded card data for the seventh solution profile;
determining a first tag references a second tag data;
adding the second tag data to the traversal criteria data; and
at least one of removing the preference data and expanding a location preference of the user profile,
wherein the entry criteria data further comprising and a property data of the user.

12. A computer readable medium storing a data structure for efficient determination of a medical solution applicable to a user, the compute readable medium comprising:
a first data entity comprising:
a unique identifier of the first data entity,
a content data of the first data entity specifying at least a name of a first solution profile and a description data describing the first solution profile,
an action function of the first data entity comprising at least one of a set of computer readable instructions and a reference to the set of computer readable instructions that when executed, initiates an action associated with the first solution profile upon receipt of an action request generated by a client device,
a first reference attribute of the first data entity storing an identifier of a second data entity to define a first reference that is unbound by hierarchical constraint within the data structure, and
at least one of a tag data and a tag reference to the tag data, the tag data comprising a first term value mapped to a second term value, wherein the second term value is selected from a medical taxonomy.

13. The computer readable medium of claim 12, wherein the second data entity comprises:
the unique identifier of the second data entity,
a content data of the second data entity specifying at least a name of a second solution profile and a description data describing the second solution profile, and
a second reference attribute of the second data entity storing an identifier of a third data entity to define a second reference that is unbound by hierarchical constraint within the data structure,
wherein the first data entity further comprises:
a reference modification data associated with the first reference attribute of the first data entity which stores data for, upon traversal of the first reference, at least one of (i) modification of the content data of the second data entity and (ii) termination of continuing traversal of the second reference to stop extraction of data from the third data entity as a derivative reference of the first data entity.

14. The computer readable medium of claim 13, further comprising:
a plurality of solution profiles each coupled to one another within a semantic data structure through a chain of one or more references unbound by hierarchical constraint, the plurality of solution profiles comprising:
a professional profile modeling a healthcare professional,
a business profile modeling a healthcare business,
at least one of a product profile modeling a healthcare product and a technology profile modeling a healthcare technology,
a service profile modeling a healthcare service, and
an educational material profile related to health.

15. The computer readable medium of claim 14, wherein:
a relation status of the first reference comprising at least one of (i) a profile unverified status designating that the first data entity is unassociated with a user profile, (ii) a relation unverified status designating that the first reference is unreviewed by a first user profile controlling the first data entity, (iii) a relation pending status designating that the first reference is to be verified by the first user profile controlling the first data entity and a second user profile controlling the second data entity, (iv) a profile verified status designating that the first data entity is controlled by the first user profile, and (v) a relation verified status designating that the first reference has been verified by the first user profile controlling the first data entity, and
a designation data of the first reference, wherein the designation data comprising at least one of an unexpressed reference and an expressed reference.

16. The computer readable medium of claim 15, wherein the first data entity further comprising:
the professional profile comprising a set of reference attributes of the professional profile referencing at least one of the business profile and the educational material profile,
the business profile comprising a set of reference attributes of the business profile referencing at least one of (i) the professional profile, (ii) the educational material profile, and (iii) the at least one of the product profile and the technology profile,
the educational material profile comprising a set of reference attributes of the educational material profile referencing at least one of (i) the business profile, (ii) the professional profile, (iii) the at least one of the product profile and the technology profile, and (iv) a different instance of the educational material profile, and
the service profile comprising a reference attribute of the service profile referencing the educational material profile.

17. The computer readable medium of claim 16, further comprising:
a specialty profile comprising a unique identifier of the specialty profile, a content data of the specialty profile, and a set of reference attributes of the specialty profile referencing two or more instances of the service profile,
a location profile comprising a unique identifier of the location profile, a content data of the location profile, and a map data comprising a geospatial coordinate of a location associated with the location profile,
wherein the at least one of the product profile and the technology profile comprising a hierarchical reference to at least one of a different instance of the product profile and a different instance of the technology profile.

18. A system for efficient database query, the system comprising:
a database server comprising:
a processor of the database server,
a memory of the database server, and
a semantic data structure comprising a plurality of solution profiles as nodes of the semantic data structure, each node coupled to one another through a chain of one or more references unbound by hierarchical constraint,
a coordination server comprising:
a processor of the coordination server,
a memory of the coordination server,
a request agent comprising computer readable instructions that when executed receive from a client device of a user an entry criteria data usable to determine an entry node of the semantic data structure, the entry criteria data comprising:
at least one of a body part value and a body organ value, and
an entry subroutine comprising computer readable instructions that when executed:
determines an entry point into one or more nodes of the semantic data structure,
query the semantic data structure to receive a first set of unique identifiers of the plurality of solution profiles each comprising a tag data matching the entry criteria data,
wherein the tag data is mapped to a term value of a medical taxonomy through a tag reference,
a filter subroutine comprising computer readable instructions that when executed:
remove a first subset of the first set of unique identifiers based on at least one of a location data associated with the user stored in a user profile of the user and a preference data of the user to result in a second set of unique identifiers; and
a card extraction module comprising computer readable instructions that when executed:
extract a card data for each data entity associated with the second set of unique identifiers,
transmit to a client device of the user the card data for each data entity associated with the second set of unique identifiers, and
a network communicatively coupling the coordination server and the database server.

19. The system of claim 18, further comprising:
the request agent further comprising computer readable instructions that when executed:
at least one of receive the preference data of the client device of the user and extract the preference data from a user profile of the user,
wherein the entry criteria data further comprising at least one of a general category value, a specific category value that is specific to at least one of the body part value and the body organ value, and a disease value, and
receive a first profile request for a first solution profile comprising a unique identifier of the first solution profile; and
the card extraction module further comprising computer readable instructions that when executed:
transmit to the client device of the user a profile data of the first solution profile,
a traversal subroutine comprising computer readable instructions that when executed:
traverse within the semantic data structure one or more references of the first solution profile to a set of solution profiles;
store a third set of unique identifiers comprising the set of solution profiles referenced through the one or more references of the first solution profile upon receiving the first profile request,
a ranking subroutine comprising computer readable instructions that when executed determine a presentation order of the card data for each data entity associated with the second set of unique identifiers on a display application running on the client device;
the filter subroutine further comprising computer readable instructions that when executed:
remove a second subset of unique identifiers from the third set of unique identifiers based on the location data associated with the user stored in the user profile and the preference data of the user to result in a fourth set of unique identifiers,
the card extraction module further comprising computer readable instructions that when executed:
transmit to the client device of the user a card data for each solution profile associated with the fourth set of unique identifiers to present semantically related data entities traversable by the user within the semantic data structure to increase a probability of the user efficiently locating the first solution profile applicable to the user,
a tag extraction routine comprising computer readable instructions that when executed:
initiate a traversal criteria data initially comprising the entry criteria data;
extract a tag data of the first solution profile upon receiving the first profile request; and
store the tag data of the first solution profile in the traversal criteria data,
wherein the third set of unique identifiers further comprising a second set of solutions profiles referencing the tag data of the first solution profile;
a priority assertion system comprising computer readable instructions that when executed:
determine that a stored reference of the first solution profile drawn to a second solution profile conflicts a stored reference of a third solution profile drawn to a second solution profile,
determine the stored reference of the first solution profile is prioritized based on at least one of a profile verification data, an entity type data, an entity size data, and a timestamp of the stored reference of the first solution profile;
determine the stored reference of the first solution profile is an expressed reference,
wherein a unique identifier of the second solution profile is included in the third set of unique identifiers for transmission of a card data of the third solution profile to a probability of modeling accuracy through expression of the stored reference of the first solution profile as the expressed reference,
an entity retention module comprising computer readable instructions that when executed:
store a reference from the user profile to a unique identifier of the first solution profile to enable the user to efficiently re-query the first solution profile;
generate at a first time a first reference snapshot data of one or more references drawn to the first solution profile saved by the user, the first reference snapshot data comprising at least one of a unique identifier of one or more data entities referencing the first solution profile and a unique identifier of one or more data entities referenced by the first solution profile;
an entity comparison routine comprising computer readable instructions that when executed:
generate at a second time a second reference snapshot data of one or more references drawn to the first solution profile saved by the user by re-traversing, upon receiving a second profile request, one or more references of the first solution profile;
wherein the second reference snapshot data comprising at least one of a unique identifier one or more data entities referencing the first solution profile and a unique identifier of one or more data entities referenced by the first solution profile;
determining the first profile data at the first time and the first profile data at the second time vary; and
transmitting a variation data to the client device of the user.

20. The system of claim 19, wherein the coordination server further comprising:
an authentication system comprising computer readable instructions that can authenticate a second user associated with controlling the first solution profile;
an authority assertion system comprising computer readable instructions that when executed:
receiving a request to delete a first reference stored within the semantic data structure, the first reference drawn from a fourth solution profile to the first solution profile to assert a relation between the fourth solution profile and the first solution profile;
compare a unique identifier of the first solution profile and a unique identifier of the fourth solution profile;
reference an authority hierarchy data;
determine the first solution profile is higher in the authority hierarchy data; and
at least one of (i) deleting the first reference in the fourth solution profile and (ii) designating the first reference as an unexpressed reference within the semantic data structure,
a card nesting subroutine comprising computer readable instructions that when executed:
extract a card data of the first solution profile;
extract a card data of a fifth solution profile;
generate a nested card data that incorporates at least a portion of the card data of the first solution profile into the card data of the fifth solution profile;
a traversal expansion routine comprising computer readable instructions that when executed:
adds a tag data to the traversal criteria data; and
at least one of removing the preference data and expanding a location preference of the user profile,
wherein the entry criteria data further comprising and a property data of the user;
wherein the card extraction module further comprising computer readable instructions that when executed:
receive a third profile request for an expanded card data, the third profile request comprising a unique identifier of a sixth solution profile;
transmit to the client device of the user the expanded card data for the sixth solution profile.

21. The system of claim 20, further comprising:
a machine learning server, comprising:
a processor of the machine learning server,
a memory of the machine learning server,
an artificial neural network comprising two or more processing nodes each comprising one or more weighted outputs;
a session management module comprising computer readable instructions that when executed:
at least one of initiates a training session and initiates a prediction session, and
receives a traversal data,
a training routine comprising computer readable instructions that when executed:
receive a fifth set of unique identifiers of solution profiles that have at least one of a set of data cards expanded by the user, a profile data requested by the user, and a set of stored references from the user profile one or more solution profiles;
input as a first precursor path comprising a fifth set of unique identifiers as a first set of one or more inputs into the artificial neural network;
receives an action request of the user associated with a seventh solution profile;
extract the unique identifier of the seventh solution profile;
input as an applicable selection at least one of the unique identifier of the seventh solution profile and a tag data of the seventh solution profile; and
adjust an output weight of one or more of the one or more weighted outputs to train the artificial neural network,
a prediction routine comprising computer readable instructions that when executed:
receive a sixth set of unique identifiers of a set of solution profiles that have at least one of a different set of data cards expanded by a different user, a different profile data requested by the different user, and a different set of stored references from a different user profile one or more different solution profiles;
input as a second precursor path the sixth set of unique identifiers as a second set of one or more inputs into the artificial neural network comprising the two or more processing nodes each comprising the one or more weighted outputs;
outputting from the artificial neural network at least one of a second tag data and the unique identifier of an eighth solution profile to predict an applicable selection of the different user.

22. The system of claim 21, wherein
the database server comprising further comprising:
a tag mapping module comprising computer readable instruction that when executed:
map the tag data comprising at least one of the body part value, the body organ value, the general category value, the specific category value, and the disease value, to a term value of the medical taxonomy through a tag reference to the tag data matching the entry criteria data,
wherein a relation status value from the first solution profile is transmitted within the card data of the first solution profile for display on the client device of the user to communicate an association strength within the semantic data structure of the first solution profile to the second solution profile.
